SEQUENCE LISTING (2 sequences)

SEQ ID NO: 1 - DNA, 26 bp, Artificial Sequence
  Forward primer
tagccaccatggatcttgaaggggac

SEQ ID NO: 2 - DNA, 36 bp, Artificial Sequence
  Reverse primer
ccttaccggttccacttccctggcgctttgttccag

(19) United States
(12) Patent Application Publication
(10) Pub. No.: US 2013/0289520 A1
(43) Pub. Date: Oct. 31, 2013

(54) TARGETED AND LIGHT-ACTIVATED CYTOSOLIC DRUG DELIVERY

(71) Applicants: Sebastien Febvay, Somerville, MA (US); Davide Maurizio Marini, Wellesley, MA (US); David E. Clapham, Wellesley, MA (US)

(72) Inventors: Sebastien Febvay, Somerville, MA (US); Davide Maurizio Marini, Wellesley, MA (US); David E. Clapham, Wellesley, MA (US)

(73) Assignees: CHILDREN'S HOSPITAL BOSTON, Boston, MA (US); MASSACHUSETTS INSTITUTE OF TECHNOLOGY, Cambridge, MA (US)

(21) Appl. No.: 13/642,210

(22) PCT Filed: Apr. 22, 2011
(86) PCT No.: PCT/US2011/033655

§ 371 Date: Jun. 7, 2013

Related U.S. Application Data
(60) Provisional application No. 61/327,634, filed on Apr. 23, 2010.

(51) Int. Cl. A61K 47/48; A61K 41/00; A61K 9/14

(52) U.S. Cl. CPC A61K 47/48015; A61K 9/143; A61K 41/0038
USPC 604/501; 514/21.2; 424/489; 424/178.1; 514/20.9

(57) ABSTRACT

The present invention provides methods and compositions for highly precise spatial and temporal control over cytosolic delivery of compounds, in particular, those compounds that would otherwise be cell-impermeable. Among other things, the present invention provides a composition for targeted drug delivery comprising a nanoparticle, a targeting moiety specific for a cell type of interest, a light-activated drug delivery system, wherein the nanoparticles are associated with the targeting moiety and the light-activated drug delivery system.

### RELATED REFERENCES

This application claims priority to U.S. provisional patent application Ser. No. 61/327,634, filed Apr. 23, 2010, the entire contents of which are herein incorporated by reference.

### BACKGROUND

The potential of nanomaterials for drug delivery has been extensively explored in recent years. Such efforts have been driven to a large extent by the need to reduce side effects in drugs via tissue-specific targeting. Beyond enhanced targetability, nanoparticle (NP)-encapsulation of drugs may also provide protection against premature degradation and enable efficient delivery of substances with poor inherent solubility or membrane permeability. A highly desirable feature of NP-based delivery platforms is precise temporal control of compound release. This can be regulated by incorporating release mechanisms triggered by environmental stimuli such as pH, temperature, or enzymatic reactions. However, there still remains a need in the art for more precise spatial and temporal control of compound release.

### SUMMARY

The present invention provides methods and compositions for highly precise spatial and temporal control over cytosolic delivery of compounds, in particular, those compounds that would otherwise be cell-impermeable. The present invention is, in part, based on the development of an inventive method for producing size-tunable (e.g., 30-200 nm), highly monodispersed nanoparticles that can be biofunctionalized and targeted to specific cell surface proteins. These nanoparticles can be loaded with a wide variety of compounds, including small molecules, proteins, nucleic acids and the like, and can mediate cytosolic release of cell-impermeable molecules via light-mediated endosomal breakage. The present invention thus combines the advantage of nanoparticle-mediated targeted delivery with highly precise temporal control of light activation. This approach may be particularly useful for expanding the pharmacological arsenal to cell-impermeable compounds to overcome multidrug resistance.

Thus, in some embodiments, the disclosure in the present application provides a composition for targeted drug delivery comprising a nanoparticle, a targeting moiety specific for a cell type of interest, a light-activated drug delivery system, wherein the nanoparticles are associated with the targeting moiety and the light-activated drug delivery system.

In some embodiments, nanoparticles with various materials (e.g., silica, metal, etc.), shape (e.g., sphere, irregular, etc.), structure (e.g., mesoporous, core/shell, etc.), size (e.g., 30 nm-200 nm), functionalization (e.g., PEGylated) and/or other properties can be used.

In some embodiments, a targeting moiety comprises an antibody or fragment thereof. In some embodiments, a targeting moiety comprises a tumor—specific antibody or fragment thereof. In some embodiments, a targeting moiety comprises an antibody specific to a multidrug resistance transporter (e.g., MDR1 (also known as P-glycoprotein), or MRP1).

In some embodiments, a light-activated drug delivery system comprises a photosensitizer and a therapeutic agent. In some embodiments, a photosensitizer can generate reactive oxygen species (ROS) upon light activation (e.g., UV, visible, infrared, X-ray, two-photon, etc.). In some embodiments, a therapeutic agent is a protein, a peptide, a nucleic acid, a chemical compound, a small molecule, or any combination thereof. In some embodiments, a therapeutic agent is an anti-cancer agent.

In various embodiments, a nanoparticle used in accordance with the present invention is associated with a targeting moiety and a light-activated drug delivery system. Association can be covalent or non-covalent.

Among other things, the present invention provides methods of treating a disease, disorder or condition using composition provided herein. Such a method can include a step of administering (e.g., intravenously, subcutaneously, or orally) a subject with a composition provided herein and exposing the composition to light.

Compositions and methods provided herein may be particularly useful in controlled drug delivery applications. In some embodiments, methods provided herein are carried out such that a drug is released in a controlled manner.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE FIGURES

The drawings are for illustration purposes only, not for limitation.

FIG. 1 shows an exemplary schematic of light-activated and targeted cytosolic delivery of membrane-impermeable compounds. (a) Antibody-functionalized nanoparticles are loaded with a model compound (the fluorescent dye Alexa546 in our experiments) and targeted to cells expressing P-gp-GFP (GFP bound to the P-glycoprotein transporter). After nanoparticles endocytosis (b), the cargo is released in the endosome (c). Exposure to light at the dye's excitation wavelength (546 nm) promotes ROS-mediated membrane damage (d), with cytosolic delivery of Alexa546 exclusively in the P-gp expressing cells.

FIG. 2 illustrates size control of exemplary mesoporous silica nanoparticles synthesized in Example 1. (a) Transmission electron micrograph of nanoparticles synthesized with 2.5 mg/mL Pluronic® F-127 as a secondary surfactant; overlay: size distribution histogram of the sample, obtained by dynamic light scattering. (b) Higher magnification transmission electron micrograph of the same NPs, showing a mesoporous structure without long-range order. (c) Relationship between the amount of secondary surfactant in the reaction mixture and nanoparticle size (hydrodynamic diameter). Each data point is the average of 3 batches synthesized independently.

FIG. 3 shows exemplary results of the application of double-surfactant templated synthesis to various types of core-mesoporous silica shell nanostructures as described in Examples 2-5. (a) Gold core. Left: transmission electron micrographs of the core-shell NPs after surfactant extraction. Middle: photograph of a suspension of the NPs. Right: intensity-based DLS size distribution (top) and zeta potential distribution (bottom) before (green trace) and after (red trace) surfactant extraction. (b) Magnetic core. Left: transmission electron micrographs of the core-shell NPs after surfactant extraction, and photograph illustrating magnetic collection of the NPs (inset). Middle: water suspension of NPs. Right: intensity-based DLS size distribution of 3 types of core-shell NPs obtained by varying synthetic conditions (top), and zeta potential distribution (bottom) before (green trace) and after (red trace) surfactant extraction. (c) Gold nanorod core. Left: transmission electron micrographs of the core-shell NPs after surfactant extraction. Middle: water suspension of NPs. Right: intensity-based DLS size distribution. (d) Quantum dot core. Left: transmission electron micrographs of surfactant-extracted NPs. Red arrows point to individual QD cores; 1 QD/NP. Inset: the core-shell NPs before and after centrifugation under UV illumination. Right: intensity-based DLS size distribution of the NPs in water.

FIG. 4 illustrates exemplary results of nanoparticle functionalization as described in Example 6. (a) Size exclusion chromatogram of a 1 mL functionalized NP sample on a Sephacryl® S-400 column (30 cm length, 1 cm diameter, flow rate of 0.4 mL/min) Elution peaks are in order: NP-conjugate, unreacted streptavidin, excess PEG-maleimide, and unbound Alexa 546 dye. (b) DLS size distributions of NPs before (red trace) and after (green trace) biofunctionalization. Typically, the limited increase in size as well as low polydispersity indicates minimal aggregation during the process of conjugation.

FIG. 5 illustrates exemplary results of loading and fluorescence properties of compounds adsorbed onto silica surface as described in Example 7. (a) Photograph of mesoporous silica NPs (100 nm diameter here) loaded with various compounds. Typically, NPs were pelleted by centrifugation at 11,000 rpm for 30 min. The compounds loaded are, from left to right: empty NPs, Tris-(2,2′-bipyridyl)-ruthenium(II) (Sigma), X-Rhod1 calcium indicator (Invitrogen). (b) Fluorescence of Alexa546 maleimide measured by plate reader (Perkin-Elmer) at various concentrations, either free (red trace) in phosphate buffered saline (PBS, 10 mM, pH 7.4), or bound to PEG-coated mesoporous silica NPs in PBS (black trace). Without being bound to any particular theory, it is believed that the exemplary results shows how association of the dye molecules with the silica matrix provides increased brightness at lower concentrations, while self-quenching occurs at higher loading rates. In this example, the amount loaded for targeting and dye delivery experiments corresponds to a concentration of 1 μM on this graph, at the upper limit of the linear range, equivalent to a 1.6% w/w ratio.

FIG. 6 illustrates exemplary results of characterization of the vesicles containing NPs after endocytosis as described in Example 8. (a) Confocal micrographs of LN-229 cells transiently transfected with the lysosomal marker, LAMP1-GFP, following incubation with PEI-PEG functionalized NPs. Images show representative cells 10 min (top), 3 hours (middle) and 20 hours (bottom) after incubation at 37° C. (b) Confocal micrographs of LN-229 cells after overnight endocytosis of streptavidin functionalized NPs via surface biotinylation. The endo-lysosomal compartment was stained with LysoTracker Blue. All scale bars are 20 microns.

FIG. 7 illustrates exemplary results of time course of PEI-PEG NPs endocytosis as described in Example 8. Confocal micrographs of live LN-229 cells transiently transfected with the early endosome marker RabS-GFP (a) and late endosome marker Rab7-GFP (b) following incubation with PEI-PEG functionalized NPs. Images show representative cells at different time points after incubation at 37° C. Early endosomes containing NPs can be observed at 60 min (arrows), while NPs can be observed in Rab-7 tagged vesicles at later time points (180 min). Scale bars are 20 microns.

FIG. 8 shows exemplary results of light-induced cytosolic release of Alexa546 loaded into mesoporous silica nanoparticles as described in Example 8. (a) Confocal micrographs of live LN-229 cells after surface biotinylation-mediated uptake of streptavidin-functionalized particles loaded with Alexa546 (60× water-immersion objective). Images were acquired before (left panels) and immediately after (right panels) exposure to light from a TRITC-filtered mercury lamp. (b) Relationship between the amount of cytosol-released Alexa546 and the amount of endocytosed NPs. Each data point in the scatter plot represents one cell. (c) Time evolution of Alexa546 fluorescence following stimulation. Fluorescence is normalized for each cell to its initial value preceding light exposure. The bars represent S.D. (n=57 cells). (d) Confocal micrographs of live LN-229 cells following overnight uptake of PEI-PEG coated NPs in the presence of calcein (0.25 mM). Images were acquired before and after 2 min light exposure as in (a). The profile plots display calcein and Alexa546 fluorescence across a representative cell before and after exposure. Scale bars are 20 microns in all images.

FIG. 9 illustrates exemplary results of cytosolic delivery of dextran. (a) Confocal micrographs of LN-229 cells after NP and dextran-FITC co-endocytosis (60× water-immersion objective). Orange NPs were loaded with Alexa546 after synthesis, while red NPs contain covalently bound Alexa633, which cannot be released. The two types of NPs were endocytosed separately and the two populations subsequently mixed and incubated with dextran. Images were acquired before (top row) and at various time points following 2 min light stimulation of the NPs (following rows). Dextran-FITC cytosolic release is observed with both NP types, with different kinetics. Scale bars are 20 microns. (b) Time evolution of NP and dextran-FITC average cell fluorescence (normalized to initial fluorescence) following light stimulation, for both Alexa546-NPs loaded cells (left, n=14 cells) and Alexa633-NPs loaded cells (right, n=20 cells). The bars represent S.D.

FIG. 10 illustrates exemplary results of cytosolic delivery of NeutrAvidin. (a) Confocal micrographs of LN-229 cells after NP and NeutrAvidin-FITC co-endocytosis (60× water-immersion objective, scale bar 20 microns). NPs were loaded with Alexa546. Images were acquired before (top row), immediately after (middle row) and 2 min following 120 s light stimulation of the NPs. Light stimulation caused rapid and massive extrusion of the loaded Alexa546 dye. The endocytosed NA-FITC conjugates, largely colocalized with NPs in the endosomal compartment before light exposure, were released in the cell cytosol as well, with slower kinetics than the dye, as expected. (b) Time evolution of Alexa546 and NeutrAvidin-FITC average cell fluorescence (normalized to initial fluorescence) following light stimulation, reflecting a significant increase for both. While the maximum fluorescence was reached before the end of light stimulation (120 s) for the Alexa dye, the maximum fluorescence for the NA-FITC conjugate is only reached after >10 min following the end of light exposure.

FIG. 11 illustrates exemplary results of cytosolic delivery of QD. Confocal micrographs of LN-229 cells after NP and QD525-streptavidin co-endocytosis. Images were acquired before (top row), and 15 min after 120 s light stimulation (60× water-immersion objective, scale bar 20 microns). In contrast to NP-released Alexa546, the QDs do not migrate into the cytosol of the cells following stimulation of the co-endocytosed NPs over a period of >30 min. This may be due to irreversible clustering of the QDs following endocytosis, or multiple streptavidin-biotin bonds for each QD, preventing separation from the endosomal compartment.

FIG. 12 shows an example in monitoring of single vesicle disruption events following light exposure as described in Example 11. Confocal micrographs of BAEC cells exposed to green light after overnight endocytosis of Alexa 546-loaded PEI-PEG MSNs. (a) Time course of Alexa 546 fluorescence at various time points following 3 s light exposure. (b) DIC images of cells before and 6 min after a 3 s light exposure, showing decreased light absorption of the NP-containing lysosomes after dye release (bar diagram, mean absorbance +/−SD, n=30 vesicles). (c) Magnified view of individual vesicles at various time points following a 3 s exposure. Two individual vesicle disruptions can be seen at ˜160 s and 201 s following exposure. (d) Fluorescence (top row) and absorbance (from DIC transmittance, bottom) profile plots along the lines defined in (c). All scale bars are 20 microns.

FIG. 13 illustrates effect of crosslinker length and nanoparticle sonication on targeting efficiency as described in Example 12. (a) Confocal micrographs of LN-229 cells stably expressing P-gp-GFP after incubation with NPs functionalized with different crosslinkers. The 2 NP types tested are identical in size (2.75 mg/mL Pluronic® F-127 in the reaction mixture), functionalized with the same amount of streptavidin (60 μg SA per 2 mg NPs), but with two different crosslinkers: LC-SMCC (Pierce Biotechnology, 15:1 molar ratio to SA, top row) and NHS-PEG-MAL 5 kDa (Rapp Polymere, 5:1 molar ratio to SA, bottom row). Images were acquired using identical settings. We speculate that the superior targeting efficiency was obtained with the longer crosslinker due to increased mobility and reduced steric hindrance for the immobilized streptavidin. (b) Confocal micrographs of LN-229 cells stably expressing P-gp-GFP after incubation with identical NPs, either sonicated for 30 s before incubation (bottom row), or without sonication (top row). All samples were imaged using a 60× water-immersion objective. Scale bars are 20 microns.

FIG. 14 illustrates exemplary results, as described in Example 12, of (a) Antibody-mediated targeting of mesoporous silica nanoparticles to P-gp. Confocal micrographs showing P-gp-GFP-expressing cells after incubation with streptavidin-functionalized, Alexa546 loaded NPs (60× water-immersion objective). The NPs were added following staining with a primary mouse anti-hMDR1 antibody and a biotinylated goat-anti-mouse secondary antibody. Plates also contained cells lacking P-gp to serve as contemporaneous controls. The bottom right graph displays the amount of NPs bound to individual cells as a function of their P-gp expression level. Each data point in the scatter plot represents one cell. The NPs used here correspond to a hydrodynamic diameter post-synthesis of ˜60 nm (b) Targeted, light-activated cytosolic release of Alexa546 into cells expressing P-gp-GFP. Streptavidin-functionalized particles (75 nm diameter) were loaded with Alexa546 and targeted to cells expressing P-gp-GFP. Confocal micrographs showing cells after overnight endocytosis of targeted NPs, before (left panel), and immediately after (right panel), light exposure. Cells were imaged using a 60× water-immersion objective. The scatter plot shows the variation in dye fluorescence following light exposure as a function of cell P-gp expression level. Each data point represents one cell. Scale bars are 20 microns in all images.

FIG. 15 illustrates exemplary results of characterization of the P-glycoprotein-GFP fusion by substrate loading, as described in Example 12. Confocal micrograph of transiently transfected cells (LN-229) expressing the construct after incubation with tetramethyl-rhodamine-esther (TMRE, 50 nM in D-PBS-glucose) for 10 min at room temperature (scale bar 20 microns). P-glycoprotein-GFP-expressing cells show complete extrusion of TMRE, while nontransfected cells display high dye loading with a typical mitochondrial distribution pattern. Peri-membrane localization of the construct is observed, and membrane localization was confirmed by live cell antibody staining to an extracellular epitope (BD, same primary antibody used for NP targeting experiments, data not shown).

### DEFINITIONS

In order for the present invention to be more readily understood, certain terms are first defined below. Additional definitions for the following terms and other terms are set forth throughout the specification.

“Associated”: As used herein, the terms “associated”, “conjugated”, “linked”, “attached”, “complexed”, and “tethered,” and grammatic equivalents, typically refer to two or more moieties connected with one another, either directly or indirectly (e.g., via one or more additional moieties that serve as a linking agent), to form a structure that is sufficiently stable so that the moieties remain connected under the conditions in which the structure is used, e.g., physiological conditions. In some embodiments, the moieties are attached to one another by one or more covalent bonds. In some embodiments, the moieties are attached to one another by a mechanism that involves specific (but non-covalent) binding (e.g. streptavidin/avidin interactions, antibody/antigen interactions, etc.). Alternatively or additionally, a sufficient number of weaker interactions (non-covalent) can provide sufficient stability for moieties to remain connected. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Biocompatible”: The term “biocompatible”, as used herein is intended to describe materials that do not elicit a substantial detrimental response in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

“Biodegradable”: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

“Hydrodynamic diameter”: The term “hydrodynamic diameter”, as used herein, generally refers to the effective diameter of a hydrated molecule (e.g., macromolecules, colloids, or particles) in solution, corresponding to the diameter of a sphere with equal mobility in solution. In some embodiments, a hydrodynamic diameter is used to describe the measured size of particles in solution. In certain embodiments, hydrodynamic diameter may be determined by dynamic light scattering size measurement. For example, Zetasizer Nano ZS instrument (Malvern) can be used to measure the hydrodynamic diameter of particles as demonstrated in the Example Section below.

“Light”: As used herein, the term “light” includes radio, microwave, infrared, the visible region, ultraviolet, X-rays, and gamma rays. The visible light has a wavelength in a range from about 380 or 400 nanometres to about 760 or 780 nm. Infrared (at lower frequencies) and ultraviolet (at higher) are not visible to human eyes.

“Monodisperse”: As used herein, the terms “monodisperse” or “monosized” refer to a collection of objects that have substantially the same size and shape when in the context of particles, and substantially the same mass in the context of polymers. Conversely, a collection of objects that have an inconsistent size, shape and mass distribution are called polydisperse. Monodisperse particles are typically synthesized through the use of template-based synthesis.

“Nanoparticle”, or “particle”: The terms “nanoparticle”, or “particles,” as used herein, refer to discrete materials, and may be used interchangeably. Such materials can be of any shape or size. In some embodiments, nanoparticles are particles having a diameter of less than 1000 nanometers (nm). Composition of particles may vary, depending on applications and methods of synthesis. Suitable materials include, but are not limited to, plastics, ceramics, glass, polystyrene, methylstyrene, acrylic polymers, metal, paramagnetic materials, thoria sol, carbon graphited, titanium dioxide, latex or cross-linked dextrans such as Sepharose, cellulose, nylon, cross-linked micelles and teflon. In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments.

“Photosensitizes”: As used herein, the term “photosensitizer” refers to any compounds that is capable of causing permeabilization of endosome membranes upon light activation. Typically, a photosensitizer generates reactive oxygen upon light activation, including, but not limited to, X-ray or UV irradiation.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present application, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. The phrase “enzyme polypeptide” refers to a polypeptide having enzymatic activity.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Therapeutic agent”: As used herein, the phrase “therapeutic agent” refers to any agent that, when administered to a subject, has a therapeutic effect and/or elicits a desired biological and/or pharmacological effect.

“Treating:” As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION OF CERTAIN EMBODIMENTS

The present invention provides a system for highly precise cytosolic drug delivery. Among other things, the present invention provides an inventive system based on the combination of specific nanoparticle-mediated targeted delivery with light-activated cytosolic cargo release.

As described in the Examples, the present inventors have developed an inventive method for synthesizing size-tunable, highly monodispersed nanoparticles, such as, monodispersed mesoporous silica nanoparticles that can be biofunctionalized and targeted to specific cell types. Highly monodispersed nanoparticles, especially with a small hydrodynamic diameters (e.g., ranging from 30-200 nm), is highly desirable to ensure efficacy of the nanoparticles and to facilitate access to sterically hindered tissues. Prior to the present invention, size control of nanoparticles such as mesoporous silica nanoparticles for drug delivery remains a challenge. For example, most mesoporous silica nanoparticles reported for biomedical applications have inherent large size and prone to aggregation in solution leading to dispersions with large hydrodynamic diameters following surfactant removal. As described in the examples, the present inventors archived accurate size control of nanoparticles (e.g., mesoporous silica nanoparticles) by introducing a secondary surfactant (e.g., Pluronic F-127) into a traditional synthesis reaction (e.g., a base-catalyzed synthesis reaction). This approach resulted in surprisingly well-dispersed nanoparticles of homogeneous size and porosity. The inventors have combined the inventive nanoparticles with photosensitizers to promote endosomal escape of their cargo and access to the cytosol upon exposure to light. As shown in the Examples section, this inventive approach has archived successful cytosolic delivery of cell impermeable compounds such as proteins and other macromolecules that the prior art methods cannot archive. In addition, this combination allows unprecedented precise temporal and spatial control over cytosolic access of the encapsulated drug in light-exposed cells, while preserving unexposed cells.

In various embodiments, compositions and methods for targeted drug delivery is disclosed.

### Nanoparticles

Typical particles suitable for use in accordance with the present invention are biocompatible. In general, a biocompatible substance is not toxic to cells. In some embodiments, a substance is considered to be biocompatible if its addition to cells results in less than a certain threshold of cell death (e.g., about 50%, about 45%, about 40%, about 35%, about 30%, about 25%, about 20%, about 15%, about 10%, about 5%, or less than about 5% cell death). In some embodiments, a substance is considered to be biocompatible if its addition to cells does not induce adverse effects.

In some embodiments, a particle used according to the present invention is biodegradable. In general, a biodegradable substance is one that undergoes breakdown under physiological conditions over the course of a therapeutically relevant time period (e.g., weeks, months, or years). In some embodiments, a biodegradable substance is a substance that can be broken down by cellular machinery. In some embodiments, a biodegradable substance is a substance that can be broken down by chemical processes. In some embodiments, a particle used according to the present invention is non-biodegradable.

In some embodiments, a particle which is biocompatible and/or biodegradable may be associated with a targeting entity and/or an agent to be delivered that is not biocompatible, is not biodegradable, or is neither biocompatible nor biodegradable. In some embodiments, a particle which is biocompatible and/or biodegradable may be associated with a targeting entity and/or an agent to be delivered is also biocompatible and/or biodegradable.

In general, particles are small enough to avoid clearance of particles from the bloodstream by the liver (e.g. particles having diameters of less than 1000 nm). Thus, in some embodiments, a particle in accordance with the present invention is any entity having a greatest dimension (e.g. diameter) of less than 1000 nanometers (nm). In some embodiments, suitable particles have a greatest dimension of less than 500 nanometers (nm). In some embodiments, suitable particles have a greatest dimension of less than about 250 nanometers (nm). In some embodiments, suitable particles have a greatest dimension (e.g. diameter) of less than about 200 nm, about 150 nm, about 100 nm, about 90 nm, about 80 nm, about 70 nm, about 60 nm, about 50 nm, about 40 nm, about 30 nm, about 20 nm, or about 10 nm. In some embodiments, particles have a greatest dimension in a range of any two values above. For example, in some embodiments, particles have a greatest dimension ranging between 5 nm and 200 nm. In some embodiments, particles have a greatest dimension ranging between 30 nm and 200 nm. In some embodiments, particles have a greatest dimension ranging between 10 nm and 100 nm. In some embodiments, particles have a greatest dimension ranging between 50 nm and 100 nm. In some embodiments, particles have a greatest dimension ranging between 30 nm and 70 nm. In some embodiments, particles have a greatest dimension ranging between 30 nm and 50 nm. In some embodiments, a greatest dimension is a hydrodynamic diameter.

In certain embodiments, particles are greater in size than the renal excretion limit. In specific embodiments, particles have diameters greater than 5 nm, greater than 10 nm, greater than 15 nm, greater than 20 nm, greater than 30 nm, greater than 40 nm, greater than 50 nm, greater than 60 nm, greater than 70 nm, greater than 80 nm, greater than 90 nm, greater than 100 nm, greater than 120 nm, greater than 150 nm, greater than 200 nm, greater than 250 nm, or larger. In general, physiochemical features of particles, including particle size, can be selected to allow a particle to circulate longer in plasma by decreasing renal excretion and/or liver clearance

In some embodiments, particles under 100 nm may be easily endocytosed in the reticuloendothelial system (RES). In some embodiments, particles under 400 nm may be characterized by enhanced accumulation in tumors. While not wishing to be bound by any theory, enhanced accumulation in tumors may be caused by the increased permeability of angiogenic tumor vasculature relative to normal vasculature. Particles can diffuse through such “leaky” vasculature, resulting in accumulation of particles in tumors.

It is often desirable to use a population of particles that is relatively uniform in terms of size, shape, and/or composition so that each particle has similar properties. In some embodiments, a population of particles with homogeneity with diameters (e.g., hydrodynamic diameters) are used. As used herein, a population of particles with homogeneity with diameters (e.g., hydrodynamic diameters) refers to a population of particles with at least about 80%, at least about 90%, or at least about 95% of particles with a diameter (e.g., hydrodynamic diameter) that falls within 5%, 10%, or 20% of the average diameter (e.g., hydrodynamic diameter). In some embodiments, the average diameter (e.g., hydrodynamic diameter) of a population of particles with homogeneity with diameters (e.g., hydrodynamic diameters) ranges from about 30 to 200 nm. In some embodiments, a population of particles with homogeneity with diameters (e.g., hydrodynamic diameters) refers to a population of particles that has a polydispersity index less than 0.2, 0.1, 0.05, 0.01, or 0.005. For example, polydispersity index of particles used in accordance with the present invention is in a range of about 0.005 to about 0.1. Without wishing to be bound by any theory, it is contemplated that nanoparticles with homogeneity (e.g., with respect to particle size) may have higher repeatability, more homogeneous delivery across the cell population, and improved targetability. Nanoparticles with homogeneity may also facilitate proper dosing of the amount of light energy required to achieve cargo release in the present application. In some embodiments, a population of particles may be heterogeneous with respect to size, shape, and/or composition.

Zeta potential is a measurement of surface potential of a particle. In some embodiments, particles have a zeta potential ranging between −50 mV and +50 mV. In some embodiments, particles have a zeta potential ranging between −25 mV and +25 mV. In some embodiments, particles have a zeta potential ranging between −10 mV and +10 mV. In some embodiments, particles have a zeta potential ranging between −5 mV and +5 mV. In some embodiments, particles have a zeta potential ranging between 0 mV and +50 mV. In some embodiments, particles have a zeta potential ranging between 0 mV and +25 mV. In some embodiments, particles have a zeta potential ranging between 0 mV and +10 mV. In some embodiments, particles have a zeta potential ranging between 0 mV and +5 mV. In some embodiments, particles have a zeta potential ranging between −50 mV and 0 mV. In some embodiments, particles have a zeta potential ranging between −25 mV and 0 mV. In some embodiments, particles have a zeta potential ranging between −10 mV and 0 mV. In some embodiments, particles have a zeta potential ranging between −5 mV and 0 mV. In some embodiments, particles have a substantially neutral zeta potential (i.e. approximately 0 mV).

Particles can have a variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc.

Particles can be solid or hollow and can comprise one or more layers (e.g., nanoshells, nanorings, etc.). Particles may have a core/shell structure, wherein the core(s) and shell(s) can be made of different materials. Particles may comprise gradient or homogeneous alloys. Particles may be composite particles made of two or more materials, of which one, more than one, or all of the materials possesses magnetic properties, electrically detectable properties, and/or optically detectable properties.

In certain embodiments of the invention, a particle is porous, by which is meant that the particle contains holes or channels, which are typically small compared with the size of a particle. For example a particle may be a porous silica particle, e.g., a mesoporous silica nanoparticle or may have a coating of mesoporous silica. Particles may have pores ranging from about 1 nm to about 50 nm in diameter, e.g., between about 1 nm and 20 nm in diameter. Between about 10% and 95% of the volume of a particle may consist of voids within the pores or channels.

Particles may have a coating layer. Use of a biocompatible coating layer can be advantageous, e.g., if the particles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, other nanoparticles that can be associated with inventive nanoparticles etc. Coatings may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties. Typically, this process is also known as functionalization. In some embodiments, particles with coating are also referred to as functionalized particles or surface treated particles.

In some embodiments, particles may optionally comprise one or more dispersion media, surfactants, release-retarding ingredients, or other pharmaceutically acceptable excipient. In some embodiments, particles may optionally comprise one or more plasticizers or additives.

A variety of different nanoparticles are of use in accordance with the invention. In some embodiments, polymeric particles may be used in accordance with the present invention. In some embodiments, particle can be or comprises inorganic polymer such as silica (SiO₂).

### Silica

In some embodiments, nanoparticles according to the invention are silica-based. For example, mesoporous silicate materials are particular useful for biomedical applications due to their biocompatibility, ease of functionalization, and large surface-to-volume ratio. Silica-based particles such as mesoporous silica particles, and any modified or hybrid particles can be of use in accordance with the present invention.

silica-based particles may be made by a variety of methods. Microemulsion procedures can be used. For example, a water-in-oil emulsion in which water droplets are dispersed as nanosized liquid entities in a continuous domain of oil and surfactants and serve as nanoreactors for nanoparticle synthesis offer a convenient approach.

Certain of these methods utilize the Stöber synthesis which involves hydrolysis of tetraethoxyorthosilicate (TEOS) catalyzed by ammonia in water/ethanol mixtures, or variations thereof. In some embodiments, silica-based particles are synthesized using known sol-gel chemistry, e.g., by hydrolysis of a silica precursor or precursors. Silica precursors can be provided as a solution of a silica precursor and/or a silica precursor derivative. Hydrolysis can be carried out under alkaline (basic) or acidic conditions. For example, hydrolysis can be carried out by addition of ammonium hydroxide to a solution comprising one or more silica precursor and/or derivatives.

Silica precursors are compounds which under hydrolysis conditions can form silica. Examples of silica precursors include, but are not limited to, organosilanes such as, for example, tetraethoxysilane (TEOS), tetramethoxysilane (TMOS) and the like. In some embodiments, silica precursor has a functional group. Examples of such silica precursors includes, but is not limited to, isocyanatopropyltriethoxysilane (ICPTS), aminopropyltrimethoxysilane (APTS), mercaptopropyltrimethoxysilane (MPTS), and the like.

In some embodiment, a silica precursor is an organosilane with a general formula R₍₄₋ₙ₎SiXₙ, where X is a hydrolyzable group such as ethoxy, methoxy, or 2-methoxy-ethoxy; R can be a monovalent organic group of from 1 to 12 carbon atoms which can optionally contain a functional organic group such as, for example, mercapto, epoxy, acrylyl, methacrylyl, and amino and the like; and n is an integer of from 0 to 4. In addition or alternatively, functional mono-, bis- and tris-alkoxysilanes for coupling and modification can be used to make silica-based particles.

In some embodiments, synthesis of various particles can involve using a secondary surfactant. Typically, a secondary surfactant is used in addition to a primary surfactant (e.g., cetyltrimethylammonium bromide (CTAB)). Secondary surfactants may be any type that is known to those of ordinary skill in the art. Exemplary surfactants include, but are not limited to, ionic surfactants, non-ionic surfactants, and combinations thereof. Examples of ionic surfactants useful in the present invention include, without limitation, sodium dodecylsulfate, sodium stearate, ammonium lauryl sulfate, and the like, and combinations thereof. Examples of non-ionic surfactants include Tween® 80 (also known as Polysorbate 80, or its chemical name polyoxyethylene sorbitan monooleate), Triton AG 98 (Rhone-Poulenc), poloxamer 407, and the like, and combinations thereof. In some embodiments, a nonionic surfactant polyol (e.g., Pluronic F-127) is used as a secondary surfactant.

It is contemplated that addition of a secondary surfactant reduces the size of the nanoparticles. As described in the Examples section, inventive synthesis methods including a secondary surfactant is able to tune the size of the resulting nanoparticles as a function of secondary surfactant concentration. Typically, increasing amounts of the secondary surfactant limit the growth of the nanoparticles, resulting in dispersions with decreasing average hydrodynamic sizes, displaying excellent reproducibility and low polydisperity.

In some embodiments, a secondary surfactant used in accordance with the present invention is in a range of about 0.01 wt % to about 1 wt % of a solution. In some embodiment, a secondary surfactant is in a range of about 0.05 wt % to about 0.5 wt %. In some embodiment, a secondary surfactant is in a range of about 0.1 wt % to about 0.25 wt %. In some embodiment, a secondary surfactant is or more than about 0.01 wt %, about 0.05 wt %, about 0.1 wt %, about 0.15 wt %, about 0.2 wt %, about 0.25 wt %, about 0.3 wt %, about 0.4 wt % or about 0.5 wt %. In some embodiment, a secondary surfactant is in a range of any two values above. In particular embodiments, 250 mg surfactant (e.g., Pluronic F-127) can be added to 100 g solution.

### Other Materials

Alternatively or additionally, other polymeric materials may be used in accordance with the present invention. For example, particles can be made of organic polymer including, but not limiting to, polystyrene, polymethylmethacrylate, polyacrylamide, poly(vinyl chloride), carboxylated poly(vinyl chloride), poly(vinyl chloride-co-vinyl acetate-co-vinyl alcohol), and combination thereof.

In some embodiments, particles are or comprise intrinsically fluorescent or luminescent particles. In certain embodiments, nanoparticles are or comprise quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible. By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source. In general, QDs are brighter and photostable than most conventional fluorescent dyes. QDs and methods for their synthesis are well known in the art (see, e.g., U.S. Pat. Nos. 6,322,901; 6,576,291; and 6,815,064; all of which are incorporated herein by reference). QDs can be rendered water soluble by applying coating layers comprising a variety of different materials (see, e.g., U.S. Pat. Nos. 6,423,551; 6,251,303; 6,319,426; 6,426,513; 6,444,143; and 6,649,138; all of which are incorporated herein by reference). For example, QDs can be solubilized using amphiphilic polymers. Exemplary polymers that have been employed include octylamine-modified low molecular weight polyacrylic acid, polyethylene-glycol (PEG)-derivatized phospholipids, polyanhydrides, block copolymers, etc.

Exemplary QDs suitable for use in accordance with the present invention in some embodiments, includes ones with a wide variety of absorption and emission spectra and they are commercially available, e.g., from Quantum Dot Corp. (Hayward Calif.; now owned by Invitrogen) or from Evident Technologies (Troy, N.Y.). For example, QDs having peak emission wavelengths of approximately 525 nm, approximately 535 nm, approximately 545 nm, approximately 565 nm, approximately 585 nm, approximately 605 nm, approximately 655 nm, approximately 705 nm, and approximately 800 nm are available. Thus QDs can have a range of different colors across the visible portion of the spectrum and in some cases even beyond.

In certain embodiments, optically detectable particles are or comprise metal particles. Metals of use include, but are not limited to, gold, silver, iron, cobalt, zinc, cadmium, nickel, gadolinium, chromium, copper, manganese, palladium, tin, and alloys thereof. Oxides of any of these metals can be used.

Certain metal particles, referred to as plasmon resonant particles, exhibit the well known phenomenon of plasmon resonance. The features of the spectrum of a plasmon resonant particle (e.g., peak wavelength) depend on a number of factors, including the particle's material composition, the shape and size of the particle, the refractive index or dielectric properties of the surrounding medium, and the presence of other particles in the vicinity. Selection of particular particle shapes, sizes, and compositions makes it possible to produce particles with a wide range of distinguishable optically detectable properties thus allowing for concurrent detection of multiple analytes by using particles with different properties such as peak scattering wavelength.

Magnetic properties of particles can be used in accordance with the present invention. Particles in some embodiments are or comprise magnetic particles, that is, magnetically responsive particles that contain one or more metals or oxides or hydroxides thereof. Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 (incorporated herein by reference) for suitable synthesis methods for certain of these particles. Additional materials that may be used in magnetic particles include yttrium, europium, and vanadium.

Nanoparticles based on other materials may be synthesized using various methods known in the art including those methods described herein. In some embodiments, a secondary surfactant may be included to control size as described herein. Exemplary secondary surfactants described above in connection with the silica-based nanoparticles may be used for synthesizing nanoparticles based on other materials.

### Functionalization

In various embodiments, the surface of particles used in accordance with the present invention can be modified, which is also referred to as functionalization or surface functionalization. As used herein, surface functionalization refers to a process of introducing chemical functional groups to a surface. In some embodiments, suitable functional groups are designed to facilitate association between nanoparticles and other entities (e.g., targeting moiety, photosensitizer, or therapeutic agents). Suitable functional groups can be introduced to the surface of particles by covalent attachment. Additionally or alternatively, coupling agents can be used with various materials/particles for functionalization. Exemplary coupling agents may include bifunctional, tri-functional, and higher functional coupling agents, which are well known in the art, such as MeSiCl₃, dioctylphthalate, polyethylene-glycol (PEG), etc. In some embodiments, particles are functionalized by covalent attachment of streptavidin onto their surface via a heterobifunctional cross-linker with a polyethylene-glycol (PEG) spacer arm. Various functionalization methods are known in the art and can be used to practice the invention.

### Nanoparticle-Mediated Targeting

In general, a nanoparticle is associated with a targeting moiety in order to target specific cell or tissue types. As used herein, the term “targeting moiety” is any entity that binds to a component associated with an organ, tissue, cell, subcellular locale, and/or extracellular matrix of interest. In some embodiments, such a component is referred to as a “target” or a “marker,” and these are discussed in further detail below. Typically, a target moiety facilitates the passive entry into target sites by reducing non-specific clearance of conjugates, and/or geometrically enhancing the accumulation of conjugates in target sites.

Additionally or alternatively, a nanoparticle is not targeted to particular tissues or cells by a targeting moiety. In certain embodiments, targeting may instead be facilitated by a property intrinsic to a nanoparticle (e.g. geometry of the nanoparticle entity and/or assembly of multiple nanoparticle entities).

### Targeting Moiety

A targeting moiety may be a nucleic acid, polypeptide, glycoprotein, carbohydrate, lipid, antibody, etc. For example, a targeting moiety can be a nucleic acid (e.g. an aptamer) that binds to a cell type specific marker. In general, an aptamer is an oligonucleotide (e.g., DNA, RNA, or an analog or derivative thereof) that binds to a particular target, such as a polypeptide. In general, the targeting function of the aptamer is based on the three-dimensional structure of the aptamer and/or target.

In some embodiments, a targeting moiety in accordance with the present invention may be a protein or peptide. In certain embodiments, peptides range from about 5 to 100, 10 to 75, 15 to 50, or 20 to 25 amino acids in size. In some embodiments, a peptide sequence can be based on the sequence of a protein. In some embodiments, a peptide sequence can be a random arrangement of amino acids. Exemplary proteins that may be used as targeting moieties in accordance with the present invention include, but are not limited to, antibodies, receptors, cytokines, peptide hormones, proteins derived from combinatorial libraries (e.g. avimers, affibodies, etc.), and characteristic portions thereof. In some embodiments, a targeting moiety may be a naturally-occurring or synthetic ligand for a cell surface receptor, e.g., a growth factor, hormone, LDL, transferrin, etc. A suitable peptide targeting moiety can be identified, e.g., using procedures such as phage display. This widely used technique has been used to identify cell specific ligands for a variety of different cell types. In some embodiments, a suitable targeting moiety is a peptide, such as an endosome disrupting peptide, translocation peptide, cell penetrating peptide, etc.

In some embodiments, a targeting moiety can be an antibody, which term is intended to include antibody fragments, characteristic portions of antibodies, single chain antibodies, etc. Synthetic binding proteins such as affibodies, etc., can be used. In some embodiments, a targeting moiety may be an antibody and/or characteristic portion thereof. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE.

As used herein, an antibody fragment (i.e. characteristic portion of an antibody) refers to any derivative of an antibody which is less than full-length. In general, an antibody fragment retains at least a significant portion of the full-length antibody's specific binding ability.

Examples of antibody or fragment thereof include, but are not limited to, CD30 antibodies, EGFR antibodies; EVI2A antibodies; Estrogen Receptor antibodies; FAM89B antibodies; IL11RA antibodies; OPRS1 antibodies; Progesterone Receptor antibodies; Transferrin Receptor antibodies; alpha 1 Fetoprotein Receptor antibodies; uPA Receptor antibodies; CA150 antibodies; CA19-9 antibodies; CA50 antibodies; CAB39L antibodies; CD22 antibodies; CD24 antibodies; CD5+CD19 antibodies; CD63 antibodies; CD66 antibodies; CTAG1B antibodies; CTAG2 antibodies; CTAGE5 antibodies; Carcino Embryonic Antigen CEA antibodies; EBAG9 antibodies; FAM120C antibodies; FLJ14868 antibodies; FMNL1 antibodies; GAGE1 antibodies; GPA33 antibodies; Ganglioside OAcGD3 antibodies; Heparanase 1 antibodies; JAKMIP2 antibodies; Lung carcinoma Cluster 2 antibodies; MAGE 1 antibodies; MUC16 antibodies; Melanoma Associated Antigen 100+/7 kDa antibodies; Mesothelin antibodies; Nestin antibodies; Neuroblastoma antibodies; Ovarian Carcinoma-associated Antigen antibodies; Prostate Specific Antigen antibodies; tumor antigens of epithelial origin antibodies; and combination thereof.

An antibody or a fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

In some embodiments, antibodies may include chimeric (e.g. “humanized”) and single chain (recombinant) antibodies. In some embodiments, antibodies may have reduced effector functions and/or bispecific molecules. In some embodiments, antibodies may include fragments produced by a Fab expression library.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

A F(ab′)2 fragment is an antibody fragment essentially equivalent to that obtained from immunoglobulins by digestion with an enzyme pepsin at pH 4.0-4.5. The fragment may be recombinantly produced.

A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

A Fab fragment is an antibody fragment essentially equivalent to that obtained by digestion of immunoglobulins with an enzyme (e.g. papain). The Fab fragment may be recombinantly produced. The heavy chain segment of the Fab fragment is the Fd piece.

In some embodiments, a targeting moiety in accordance with the present invention may be a small molecule. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol. One of ordinary skill in the art will appreciate that any small molecule that specifically binds to a desired target can be used in accordance with the present invention.

In some embodiments, a targeting moiety in accordance with the present invention may comprise a carbohydrate (e.g. glycoproteins, proteoglycans, etc.). In some embodiments, a carbohydrate may be a polysaccharide comprising simple sugars (or their derivatives) connected by glycosidic bonds, as known in the art. Such sugars may include, but are not limited to, glucose, fructose, galactose, ribose, lactose, sucrose, maltose, trehalose, cellobiose, mannose, xylose, arabinose, glucoronic acid, galactoronic acid, mannuronic acid, glucosamine, galatosamine, and neuramic acid. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In some embodiments, the carbohydrate may be aminated, carboxylated, acetylated and/or sulfated. In some embodiments, hydrophilic polysaccharides can be modified to become hydrophobic by introducing a large number of side-chain hydrophobic groups.

In some embodiments, a targeting moiety in accordance with the present invention may comprise one or more fatty acid groups or salts thereof (e.g. lipoproteins). In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid group may be a C₁₅-C₂₅ fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

### Target or Marker

In some embodiments, targeting moieties bind to a “target” or “marker” associated with an organ, tissue, cell, extracellular matrix component, and/or intracellular compartment. Typical targets or markers include cell surface proteins, e.g., receptors. Exemplary receptors include, but are not limited to, the transferrin receptor; LDL receptor; growth factor receptors such as epidermal growth factor receptor family members (e.g., EGFR, HER-2, HER-3, HER-4, HER-2/neu) or vascular endothelial growth factor receptors; cytokine receptors; cell adhesion molecules; integrins; selectins; CD molecules; etc.), a transmembrane protein, an ion channel, and/or a membrane transport protein.

In some embodiments, a suitable target or marker is associated with a specific developmental stage or a specific disease state. In certain embodiments, a marker is a tumor marker. The marker may be a polypeptide that is expressed at higher levels on dividing than on non-dividing cells. Nucleolin is an example. The peptide known as F3 is a suitable targeting agent for directing a nanoparticle to nucleolin (Porkka et al., 2002, Proc. Natl. Acad. Sci., USA, 99:444; Christian et al. 2003, J. Cell Biol., 163:871; both of which are incorporated herein by reference). For example, conjugating nanoparticles (QDs) with peptide F3 can be performed to improve nanoparticle uptake by tumor cells. The marker can be a molecule that is present exclusively or in higher amounts on a malignant cell, e.g., a tumor antigen. For example, prostate-specific membrane antigen (PSMA) is expressed at the surface of prostate cancer cells. In certain embodiments, the marker is an endothelial cell marker.

In some embodiments, a target is more prevalent, accessible, and/or abundant in a diseased tissue or cell than in a healthy tissue or cell. In some embodiments, a suitable target is preferentially expressed in tumor tissues as compared to normal tissues. In some embodiments, a suitable target is preferentially expressed in diseased (e.g., tumor or cancer) tissues or cells that have developed drug resistance. In some embodiments, a suitable target is preferentially expressed in diseased (e.g., tumor or cancer) tissues or cells that have developed multi-drug resistance. In most cases, drug resistance, especially multi-drug resistance, results from the expression of membrane transporters that actively extrude cell-permeable cytotoxic compounds. Thus, in some embodiments, suitable targets are one or more such membrane transporters. One well characterized MDR transporter is P-glycoprotein (P-gp), a 170 kD member of the adenosine triphosphate (ATP)-binding cassette (ABC) superfamily that can extrude a wide spectrum of compounds. In some embodiments, a suitable target for the invention is P-glycoprotein.

### Light-Activated Drug Delivery System

Nanoparticles described herein can be associated with a targeting moiety and a light-activated delivery system. As used herein, a light-activated drug delivery system generally includes a photosensitizer and a therapeutic agent. In certain embodiments, a light-activated drug delivery system include a photosensitizer that serves as a therapeutic agent as well. In some embodiments, a nanoparticle can be functionalized (e.g., surface functionalized by adsorption or covalently bonding) or “doped” or “loaded” with a light-activated drug delivery system.

### Photosensitizer

A sensitizer is a compound that can be induced to generate a reactive intermediate or species such as singlet oxygen. In some embodiments, a sensitizer used in accordance with the invention is a photosensitizer. As used herein, photosensitizers are sensitizers that can be induced by exposure to light to generate a reactive oxygen species. As used herein, the term “light” includes radio, microwave, infrared, the visible region, ultraviolet, X-rays, gamma rays and two-photon. In some embodiments, a photosensitizer is a dye or an aromatic compounds. In some embodiments, a photosensitizer is a compound having multiple conjugated double or triple bonds. In some embodiments, photosensitizers can release a reactive oxygen species (ROS) by heat.

Other sensitizers included within the scope of the invention are compounds that can be induced by heat, ionizing radiation, or chemical activation to generate a reactive oxygen species (ROS) (e.g., singlet oxygen).

Compositions of the present invention may include one or more photosensitizers. Desired characteristics for a photosensitizer may include at least one or more of the following characteristics: good absorption of light in a wavelength that penetrates tissue to the desired depth, compound sensitive to pH-inactive, lower activity or activity destroyed at the pH characteristic of normal tissues, but active or higher activity at the pH of the cells or organisms to be treated; compound cleared from the body quickly and if a compound is intended to treat solid tumors it may have the ability to function either in the presence and/or absence of oxygen to address the problem of tumor cell hypoxia. A photosensitizer can have low dark cytotoxicity, and excellent photopotentiation upon light illumination.

In some embodiments, photosensitizers can absorb light in the wavelength range of about 200 to about 1,100 nm, about 300 to about 1,000 nm, or about 450 to about 950 nm, with an extinction coefficient at its absorbance maximum greater than about 500 M⁻¹ cm⁻¹, about 5,000 M⁻¹ cm⁻¹, or about 50,000 M⁻¹ cm⁻¹, at the excitation wavelength. The lifetime of an excited state produced following absorption of light in the absence of oxygen can be at least about 100 nanoseconds, or, at least about 1 millisecond. In some embodiments, a lifetime is sufficiently long to mediate endosome rupture for improved cytosolic delivery in accordance with the present invention.

A large variety of light sources are available to photo-activate photosensitizers to generate ROSs. For example, a photosensitizer used in accordance with the present invention can be activated by UV, visible, infrared light or X-ray. In certain embodiments, a two-photon excitation can be used.

In some embodiments, a photosensitizer are activated by visible, near IR or IR light. Exemplary photosensitizers include, but are not limited to Alexa546, Alexa633, AIPcS₂ₐ TPPS₂ₐ, Rose bengal, zinc phthalocyanine, merocyanine, fluorescein, methylene blue, malachite green, protoporphyrin IX, indocyanine green, copper phthalocyanine and combination thereof.

In some embodiments, a photosensitizer are activated by X-ray or UV irradiation. Exemplary photosensitizers include, but are not limited to protoporphyrin IX, amifostine, clofibrate, efaproxiral, pentoxifylline, metronidazole, misonidazole, etanidazole, pimonidazole, nimorazole, sanazole, nitracrine, tirapazamine, RUS1069, RB6145, capecitabine, AQ4N, temozolomine, AG14361, lisofylline, gemcitabine, camptothecin, celecoxib, L778,123, vandetanib, gefitinib, buthionine sulfoximine, and combination thereof.

Both polychromatic and monchromatic sources may be used as long as the source is sufficiently intense to produce enough ROSs in a practical time duration. The length of the irradiation is dependent on the nature of a photosensitizer, the power of the source of irradiation, and its distance from the sample, and so forth. In general, a period for irradiation may be less than about a microsecond to as long as about 10 minutes, usually in a range of about one millisecond to about 60 seconds. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; etc.

### Therapeutic Agents

In theory, any agents including, for example, therapeutic agents (e.g. anti-cancer agents), cytotoxic agents, diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be delivered by the disclosed nanoparticles.

In some embodiments, compositions and methods in accordance with the present invention are particularly useful for delivery of at least one therapeutic agent. Exemplary agents include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, a therapeutic agent to be delivered is an agent useful in cancer treatment (e.g., an anti-neoplastic agent).

In some embodiments, a therapeutic agent is a small molecule and/or organic compound with pharmaceutical activity. In some embodiments, a therapeutic agent is a clinically-used drug. In some embodiments, a therapeutic agent is an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, etc.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

In some embodiments, a therapeutic agent may be a nucleic acid. In some embodiments, a therapeutic agent is or comprises an oligonucleotide. Exemplary oligonucleotides include, but are not limited to, antisense nucleic acids, ribozymes, siRNA, microRNA, aptamer and combination thereof. Nucleic acids containing a variety of different nucleotide analogs, modified backbones, or non-naturally occurring internucleoside linkages can be used as well.

In some embodiments, a therapeutic agent may be a protein or peptide. In certain embodiments, peptides range from about 5 to about 40, about 10 to about 35, about 15 to about 30, or about 20 to about 25 amino acids in size. Peptides from panels of peptides comprising random sequences and/or sequences which have been varied consistently to provide a maximally diverse panel of peptides may be used.

In some embodiments, a therapeutic agent may be an antibody. In some embodiments, antibodies may include, but are not limited to, polyclonal, monoclonal, chimeric (i.e. “humanized”), single chain (recombinant) antibodies. In some embodiments, antibodies may have reduced effector functions and/or bispecific molecules. In some embodiments, antibodies may include Fab fragments and/or fragments produced by a Fab expression library.

Additionally or alternatively, an agent to be delivered is a diagnostic agent. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Those skilled in the art will recognize that this is an exemplary, not comprehensive, list of agents that can be delivered using compositions and methods in accordance with the present invention. Any agent may be associated with nanoparticles for targeted delivery in accordance with the present invention.

### Use and Applications

Compositions and methods according to the present invention can be used to deliver various therapeutic or diagnostic agents into cytosols of specific cells. Compositions and methods according to the present invention are particularly useful for cytosolic delivery of compounds that would otherwise be cell-impermeable and/or for precise spatial and temporal control over cytosolic delivery. For example, nanoparticles may be delivered to a specific tissue of interest (e.g., a tumor) or cells (even single cell) of interest. In some embodiments, release of therapeutic agents can be precisely controlled. For example, nanoparticle compositions may be delivered and sequestered within an endosome. In some embodiments, compositions are sequestered in endosomal compartments for a period of minutes, hours, days, weeks, or months. Compositions may then be released from the endosome when exposed to appropriate light. Illumination of light (e.g., UV, visible, near-infrared, X-ray, etc.) can be used in a highly controlled manner to trigger a light-activated highly controlled drug delivery in cytosols.

Compositions and methods, according to the present invention, may be used for treating various diseases, disorders and/or conditions, in particular, various cancer or tumors. Exemplary cancer types include, but are not limited to, prostate, bladder, lung, liver, breast, osteosarcoma, pancreatic, colon, skin, melanoma, testicular, colorectal, urothelial, renal cell, hepatocellular, leukemia, lymphoma, ovarian cancer, central nervous system malignancies, retinoblastoma, eye-related cancers.

In some embodiments, compositions and methods of the present invention are used to target drug resistant cells (e.g., cancer or tumor cells), in particular, multi-drug resistant (MDR) cells. Almost half of human tumors develop MDR, whereby exposure to a chemotherapeutic agent triggers simultaneous resistance to a wide spectrum of different compounds, even to those to which the cell had never been exposed. In most cases, MDR results from the expression of membrane transporters that actively extrude cell-permeable cytotoxic compounds. One exemplary MDR transporter is P-glycoprotein (P-gp), a 170 kDa member of the adenosine triphosphate (ATP)-binding cassette (ABC) superfamily that can extrude a wide spectrum of compounds. Prior to the present invention, such lack of substrate specificity makes this clearance pathway difficult to circumvent. Inventive compositions and methods provided by the present invention allow cytosolic delivery of cell-impermeable compounds, which are generally not P-gp substrates thereby bypassing MDR and effectively killing those MDR tumor cells.

Nanoparticle-based compositions can be formulated and administered to a subject using any amount and any route of administration effective for treating a disease, disorder, and/or condition. Optimal amount will vary from subject to subject, depending on the species, age, and general condition of the subject, the severity of the infection, the particular composition, its mode of administration, its mode of activity, and the like.

Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Compositions (e.g., pharmaceutical composition in accordance with the present invention) may be administered to animals, such as mammals (e.g., humans, domesticated animals, cats, dogs, mice, rats, etc.). In some embodiments, compositions are administered to humans. In some embodiments, compositions of the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, compositions are administered by systemic intravenous injection, regional administration via blood and/or lymph supply, and/or direct administration to an affected site (e.g. a therapeutic implant, such as a hydrogel).

In some embodiments, provided compositions and methods are used in conjunction with a surgery.

Compositions in accordance with the present invention may be administered either alone or in combination with one or more other therapeutic agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures.

### EXAMPLES

### Example 1

### Synthesis and Characterization of Nanoparticles

This example demonstrates that various methods may be used to synthesize and/or characterize nanoparticles suitable for the present invention. In particular, experiments described herein allow accurate size control and homogeneity with hydrodynamic diameters of nanoparticle. Exemplary methods are described in detail below.

An exemplary modified synthesis procedure was developed based on the co-condensation of tetraethyl ortho-silicate (TEOS) and mercaptopropyltrimethoxysilane (MPTMS), by introducing a secondary surfactant (e.g., Pluronic® F-127) into the reaction.

Specifically, before synthesis, all glassware was washed overnight in a base bath and thoroughly rinsed using ultrapure water. In a typical synthesis, 200 mg of cetyltrimethylammonium bromide (CTAB, Sigma) and 250 mg of Pluronic® F-127 (Sigma) were placed in an Erlenmeyer flask and dissolved in 100 mL of ultrapure water. To this solution, 0.7 mL of 2 M NaOH were added and the mixture was placed in a water bath at 80° C. and magnetically stirred at 400 rpm. In a polypropylene tube (Falcon), a mixture of 1 mL of tetraethyl ortho-silicate (TEOS, Sigma) and 0.2 mL of mercaptopropyl trimethoxysilane (MPTMS, Sigma) was freshly prepared and rapidly injected into the stirring reaction mixture. The solution turned from clear to slightly opaque within a few minutes, indicating the formation of nanoparticles (NPs), and stabilized after ˜20 min. The reaction was allowed to continue for 2 h at 80° C., cooled to room temperature, and filtered at 0.2 μm (Nalgene).

Extraction of the CTAB templating surfactant from the pores was performed by adding to the filtered particles an equal volume of ethanol, followed by hydrochloric acid (36% w/w) up to a final concentration of 0.1 M. The resulting mixture was magnetically stirred at 60° C. overnight. The NP suspension was then transferred to a dialysis cassette (Slide-A-Lyzer 7,000 MWCO, Pierce Biotechnology) and dialyzed against 50% ethanol/water at 40° C. overnight, then against purified water (18 MΩ) for another 12 h. Removal of the secondary surfactant, Pluronic® F-127, was performed by repeated concentration and dilution cycles in 50% ethanol using Amicon® Ultra-15 centrifugal filter units (Millipore). An optimal number of washing cycles removes enough Pluronic® F-127 to allow access to thiol groups for streptavidin attachment, but does not completely remove the protective layer, thus avoiding NP aggregation during the following conjugation steps.

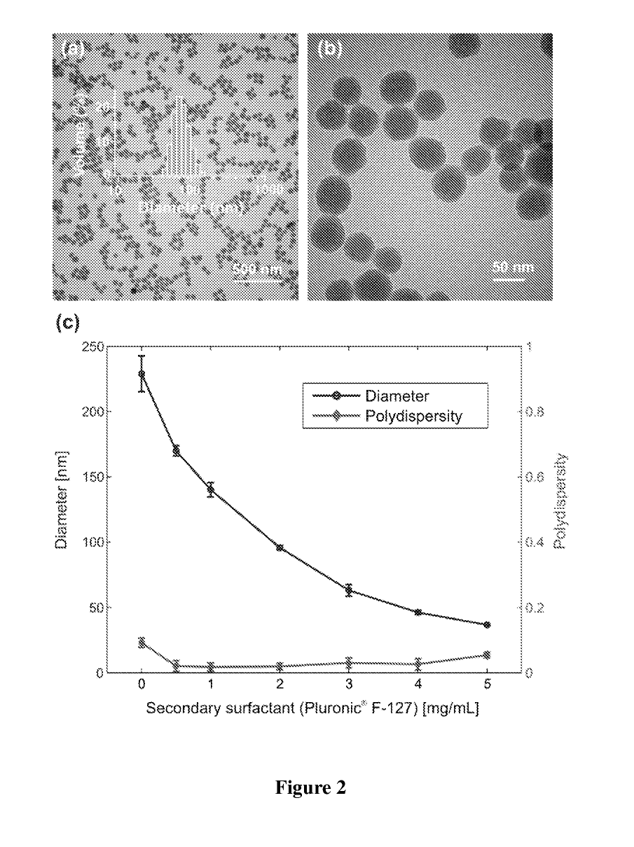

Nanoparticles obtained by introducing a secondary surfactant in a base-catalyzed synthesis reaction yielded well-dispersed NPs of homogeneous size and porosity (FIG. 2), although the mesopore network in each particle appears to be less organized than in the absence of a secondary surfactant. The graph in FIG. 2 demonstrates the ability to tune the size of the resulting NPs as a function of secondary surfactant concentration (Pluronic® F-127), as measured by dynamic light scattering (DLS, Zetasizer Nano ZS, Malvern). Increasing amounts of the non-ionic secondary surfactant limit the growth of the NPs, resulting in dispersions with decreasing average hydrodynamic sizes, displaying excellent reproducibility and low polydispersity.

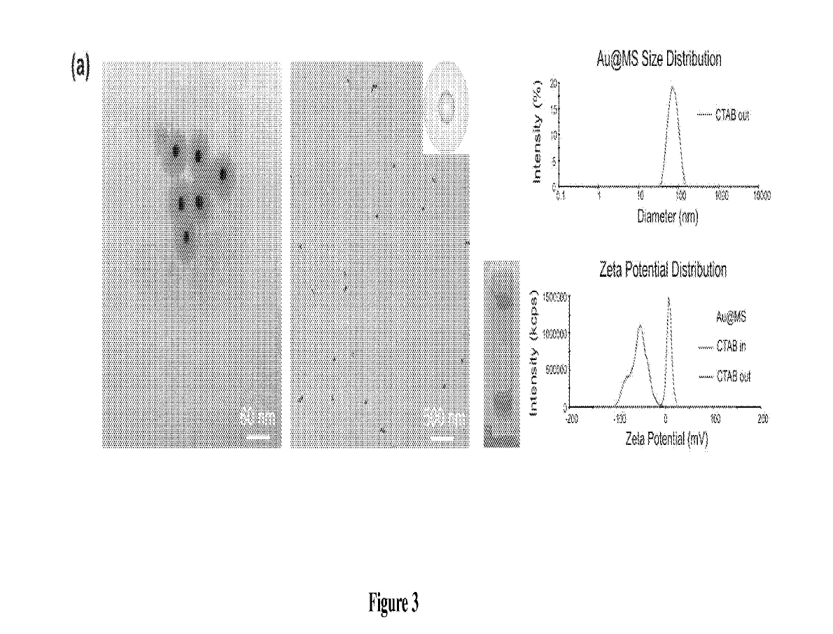

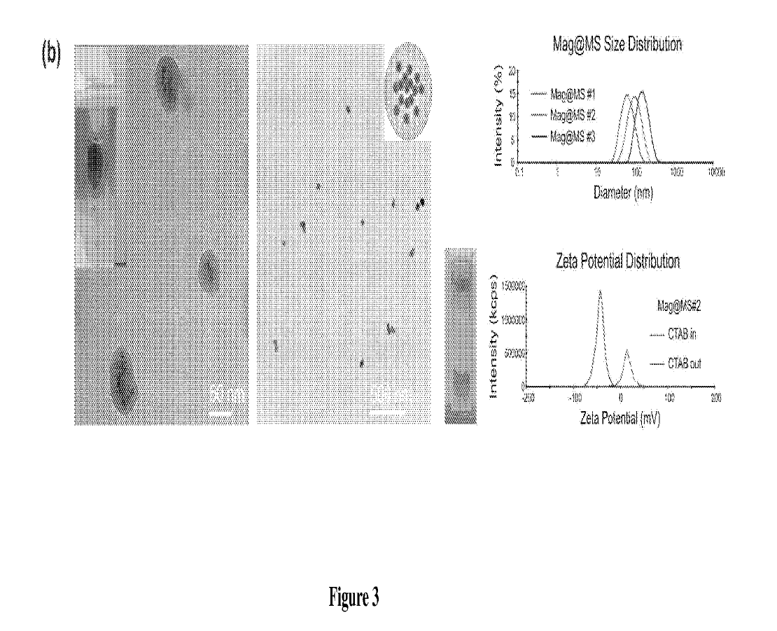

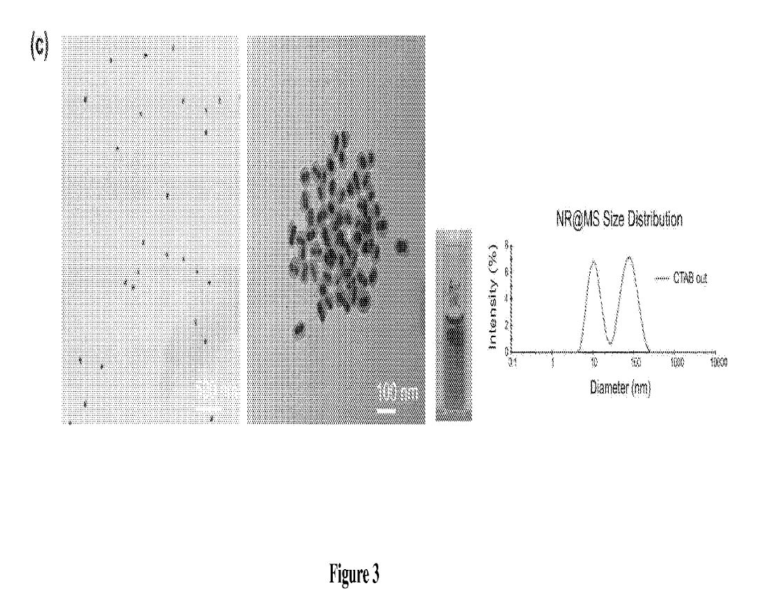

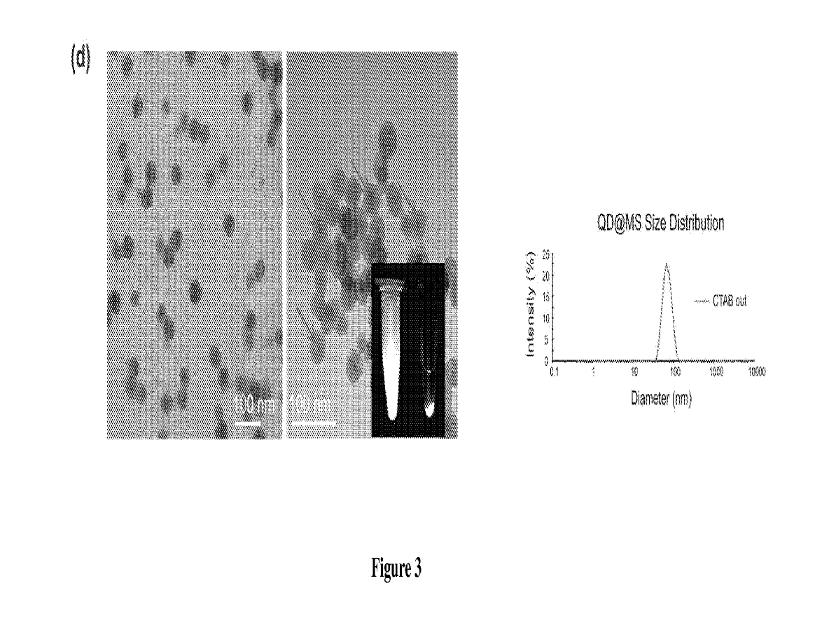

Exemplary synthetic route above could be extended to co-condensation of other organosilicate precursors beside MPTMS, such as aminopropyltriethoxysilane (APTES) and the like. In addition, a variety of composite particles could be synthesized by introducing external particles (such as gold nanospheres and nanorods, magnetic NPs, and quantum dots) into the reaction mixture and varying the amount and type of secondary surfactant, to yield core-shell nanostructures as described in Examples 2-5 (FIG. 3).

For those smaller and delicate NPs, centrifugation was difficult and aggregation-prone. A procedure based on dialysis followed by membrane filtration was used to purify the particles and produced excellent results, preserving the small hydrodynamic size of the dispersed NPs throughout the extraction and washing steps.

For characterization, dynamic light scattering size and zeta potential measurements were performed on dilute suspensions in ultrapure water after pH neutralization using a Zetasizer Nano ZS instrument (Malvern). For functionalized NPs, the measurements were performed on dilute suspensions of NPs in PBS.

### Example 2

### Synthesis of Gold-Core/Mesoporous-Silica-Shell Nanoparticles

This Example and Examples 3, 4 and 5 demonstrate that a variety of composite particles could be synthesized by introducing external particles (such as gold nanospheres and nanorods, magnetic NPs, and quantum dots) into the process described in Example 1 to yield core-shell nanostructures.

For example, gold nanoparticles were synthesized using the standard Turkevitch method (Turkevitch, J., Stevenson, P. C. & Hillier, J. Nucleation and growth process in the synthesis of colloidal gold. Discuss. Faraday Soc. 11, 55-75 (1951). All glassware was washed in a basic solution (NaOH/Ethanol) or with aqua regia (⅔HCl, ⅓ HNO₃). 200 mL of a 0.25 mM solution of HAuCl₄ was boiled while magnetically stirring at ˜440 rpm. To this solution, 3.4 mL of a 50 mM aqueous solution of trisodium citrate was added to obtain nanoparticles of ˜15 nm diameter (determined by TEM) and an estimated concentration of ˜2 nM. This nanoparticle solution was then used in the coating reaction.

200 mg of CTAB and 500 mg of Pluronic® F68 (Sigma) were dissolved in 100 mL of gold nanoparticles. 0.7 mL of 2 M NaOH was added to the mixture, which was then placed in a water bath at 80° C. under magnetic stirring at 400 rpm. In a separate tube, a mixture of 1 mL of TEOS and 0.2 mL of MPTMS was freshly prepared and rapidly injected into the gold nanoparticle mixture under vigorous stirring. The solution turned from clear red to slightly opaque within a few minutes indicating the formation of nanoparticles, and stabilized after approximately 20 min. The reaction was allowed to continue for 2 h at 80° C. The mixture was then allowed to cool to room temperature and was filtered at 0.2 μm (Nalgene).

### Example 3

### Synthesis of Gold-Nanorod-Core/Mesoporous-Silica-Shell Nanoparticles

Gold nanorods (NR) were synthesized following the method developed by Jana and Murphy (Jana, N. R., Gearheart, L. & Murphy, C. J. Wet chemical synthesis of high aspect ratio cylindrical gold nanorods. J. Phys. Chem. B 105, 4065-4067 (2001)). Briefly, 14.5 g of CTAB were dissolved in 200 mL of ultrapure water (18 MΩ) and kept at 37° C. with magnetic stirring at 120 rpm. A 5 mL aliquot was taken from this solution for the preparation of seeds. To the CTAB solution, 6 mL of 4 mM AgNO₃ aqueous solution was added, followed by 200 mL of 1 mM HAuCl₄ aqueous solution, and by 2.25 mL of a 0.1 M ascorbic acid solution.

Preparation of the gold seeds: 5 mL of 0.5 mM HAuCl₄ (Sigma) was added to 5 mL of the original CTAB solution, followed (under continuous stirring) by 0.6 mL of ice-cold 10 mM NaBH₄ (Sigma). To induce formation of gold nanorods, 400 μL of gold seeds were then added to the growth solution while stirring at 200 rpm.

Mesoporous silica coating procedure: 40 mL of the previously prepared gold NR solution were washed ×3 by centrifugation, and re-suspended each time in a 2 mg/mL solution of CTAB in water, the final CTAB concentration used in the coating reaction. The washed NRs were mixed with 80 mL of a 2 mg/mL CTAB solution in ultrapure water, to which 440 mg of Pluronic® F68 was added. After addition of 0.7 mL of 2 M NaOH, the mixture was heated to 80° C., and the pre-mixed silicate precursors (1 mL TEOS+0.2 mL MPTMS) were rapidly injected. The resulting particles were allowed to cool to room temperature, passed through a 200 nm filter, and CTAB extracted in a 1% HCl in 50% ethanol/water solution overnight, followed by several washes in 50% ethanol/water. The large amount of silicate precursors used in the synthesis relative to the amount of nanorods results in the formation of multiple small pure silica nanoparticles in addition to coated NRs. However, these additional particles are easily removed after a few rounds of centrifugation/resuspension (FIG. 3).

### Example 4

### Synthesis of Magnetic-Core/Mesoporous-Silica-Shell Nanoparticles

Mixed magnetite/maghemite nanoparticles were obtained via aqueous synthesis following the Massart process. Briefly, 10 mL of 1 M FeCl₃ in water and 2.5 mL of 2 M FeCl₂ in 2 M HCl were added to 125 mL of a 0.7 M ammonia aqueous solution under magnetic stirring. The reaction was allowed to proceed for 2 h, after which the brown precipitate was magnetically decanted, washed with deionized water, and peptized in 1 M tetramethylammonium hydroxide (125 mL).

Mesoporous silica coating procedure: 20 mL of the previously synthesized magnetic cores were centrifuged at 14,000 rpm for 1 h on a tabletop centrifuge and resuspended in 20 mL of a 14 mM tetramethylammonium hydroxide solution. In order to eliminate aggregated particles, the resuspended NPs were centrifuged again at 10,000 rpm for 20 min, and the supernatant was collected. 20 mL of magnetic cores were slowly introduced into an Erlenmeyer flask containing 80 mL of a 14 mM water solution of tetramethylammonium hydroxide (replacing NaOH in the mixture), 200 mg of CTAB, and 500 mg of Pluronic® F68. The mixture was heated to 80° C., after which 1 mL of TEOS premixed with 0.2 mL of MPTMS was rapidly injected. The reaction was allowed to proceed at 80° C. for 2 h. After cooling to room temperature, the NPs were filtered at 0.2 um. Surfactant extraction was carried out as described for other particle types.

Varying the amount of magnetic cores introduced in the synthesis, as well as the amount of secondary surfactant, allowed formation of NPs of varying diameter (FIG. 3).

### Example 5

### Synthesis of Quantum-Dot-Core/Mesoporous-Silica-Shell Nanoparticles

Fort Orange quantum dots (QD) with emission at 600 nm were purchased from Evident as a 10 mg/mL toluene solution. In a typical reaction, 100 μL of the QD solution (0.1 mg) was transferred to methanol by addition of 400 μL of 3:1 methanol:isopropanol mixture followed by centrifugation, drying and sonication into 200 μL of pure methanol.

200 μL QDs in CHCl₃ were added to 4 mL of a 10 mg/mL CTAB solution in water under stirring and heated to 60-70° C. to evaporate the methanol from the microemulsion. 2 mL (0.05 mg QDs) of the resulting solution were added to 1.5 mL of a 20 mg/mL solution of Pluronic® F127 in water, 6.5 mL water and 70 μL of 2 M NaOH. The mixture was sonicated and centrifuged for 30 min at 12,000 rpm to remove QD-surfactant aggregates, heated to 80° C., and 100 μL TEOS were added. After 30 min, another 70 μL NaOH were added, followed by 100 μL TEOS and 20 μL MPTMS. The reaction was allowed to proceed for an additional 2 h, after which the NPs were allowed to cool to room temperature and filtered at 0.2 μm.

### Example 6

### Functionalization of Nanoparticles

The experiment described in this example demonstrates that nanoparticles may be functionalized for various applications.

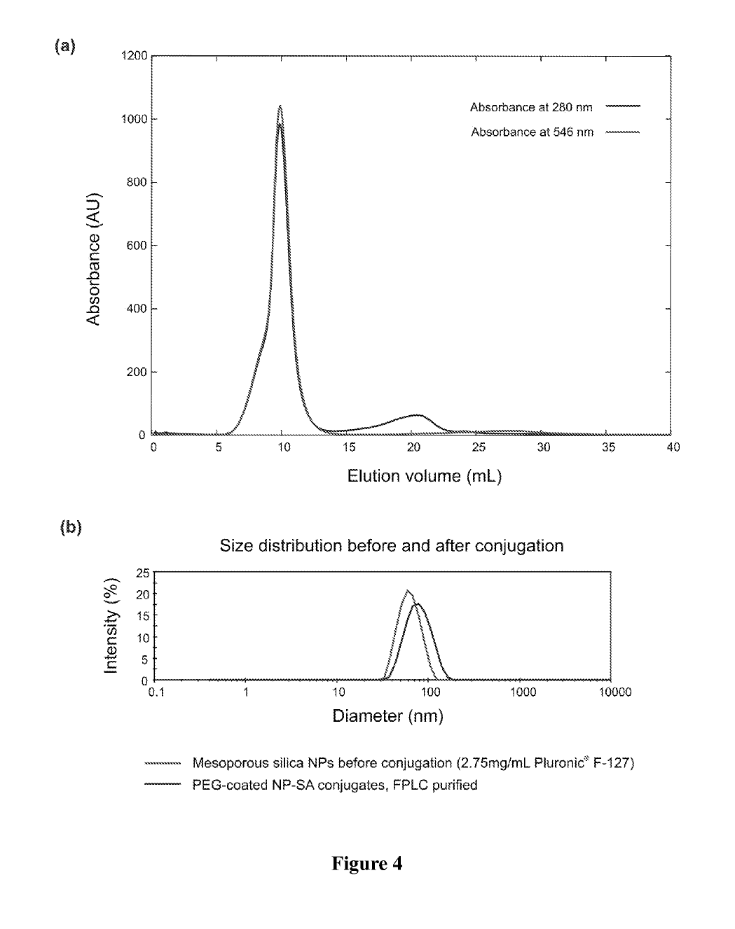

Typically, following surfactant template removal, the particles were functionalized with, e.g., streptavidin by covalent attachment via a hetero-bifunctional crosslinker with a 5 kDa poly-ethylene-glycol (PEG) spacer arm (NHS-PEG-maleimide, JenKem), or with shorter arm crosslinkers such as LC-SMCC (Pierce Biotechnology). Streptavidin (5 mg/mL) was typically first reacted with the crosslinker in phosphate buffered saline (PBS) at a molar ratio of approximately 10:1 crosslinker molecules per streptavidin for 10 min, then transferred into the NP solution (100 μg SA/2 mg NPs), and reacted overnight at 4° C. To protect the particles from aggregation in physiological buffers, 5 kDa PEG-maleimide (Rapp Polymere, Germany) freshly dissolved in water was added to the nanoparticles solution to a final concentration of 4 mg/mL, and incubated for >2 h at 4° C. Finally, PBS 10× concentrate was added to the NP suspension to reach physiological osmolarity (300 mOsm). Unbound streptavidin was removed by size-exclusion chromatography (Sephacryl® S-400 column, GE Healthcare). For all diameters, a 5 kDa PEG coat added following streptavidin attachment was important to ensure stability and agglomeration-free transfer to physiological buffers (FIG. 4). At this point, particles could be loaded with the desired cargo molecule. Typically, NPs were loaded with Alexa 546-maleimide (Invitrogen) at 50 μM by overnight incubation, resulting in both covalently immobilized dye as well as hydrolyzed dye adsorbed non-specifically onto the silica surface and within the pores. A second round of size-exclusion chromatography removed unbound Alexa546.

Similar methods can be used to functionalize nanoparticles with various polymeric or protein-based coatings (including, but not limited to, Pluronic® F-127 itself, Synperonic® PE-F68, bovine α- and β-casein and polyethylene-imine). Such functionalization enabled successful transfer of nanoparticles to physiological buffers. In some case, such functionalization may result in higher non-specific interactions between NPs and cells or glass coverslips, which may be desirable for some applications. For example, particles coated with a mixture of polyethyleneimine (25 kDa) and PEG (PEI-PEG NPs) were synthesized to mediate high non-specific cellular uptake due to high electrostatic binding of the NPs to the negatively charged cell surface.

For preparation of the PEI-PEG coated particles, the surfactant washed NPs (2 mg/mL) were incubated with 25 ug/mL of 25 kDa polyethyleneimine (Sigma, titrated to pH 7), followed by addition of 5 kDa PEG-maleimide (3 mg/mL) as detailed above.

### Example 7

### Loading of Molecules to Nanoparticles

The experiment described in this example demonstrates that a variety of molecules including photosensitizers, therapeutic agents and other compounds may be loaded onto various nanoparticles.

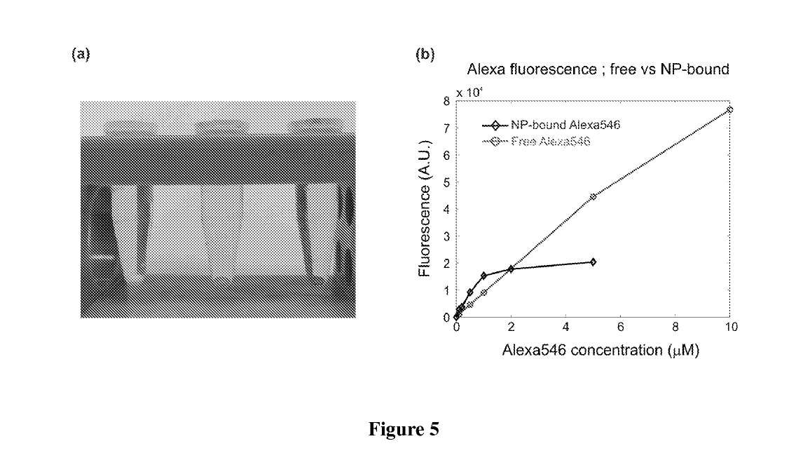

Briefly, after synthesis, purification and functionalization, nanoparticles could be loaded with a variety of molecules. In some cases, the amount of molecules loaded to the nanoparticles is equivalent to up to 12% w/w ratio (FIG. 5). In this example, the cell-impermeable dye, Alexa 546 (Invitrogen), was chosen as a model drug for its bright fluorescence. Following surface functionalization, NPs were incubated with an excess of thiol-reactive Alexa 546-maleimide in order to covalently label the free surface thiol groups, as well as saturate the silica surface and the mesopores with hydrolyzed dye. Dye loading in the nanoparticles (˜1.6% w/w) was optimized to maximize particle brightness.

For example, NPs were obtained at 2.75 mg/mL Pluronic® F-127 in the reaction mixture. Particles dissolved at 60 μg/mL were incubated overnight with the dye, after which the unbound dye was removed by centrifugation and re-suspension. The amount of immobilized dye was calculated by subtracting the supernatant fluorescence from the total amount initially added. Exemplary results illustrating the loading and fluorescent properties of compounds adsorbed onto the surface of nanoparticles are shown in (FIG. 5).

### Example 8

### Cytosolic Cargo Delivery Using Nanoparticles

This example demonstrated that nanoparticles described herein may be used to deliver cargo, including both small or macromolecules, to the cytosol.

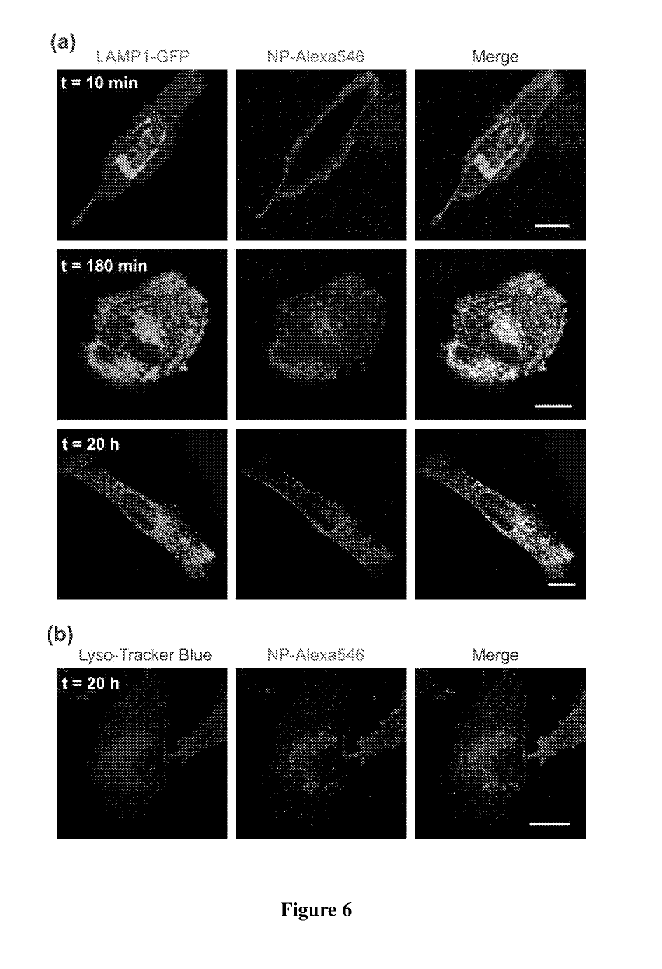

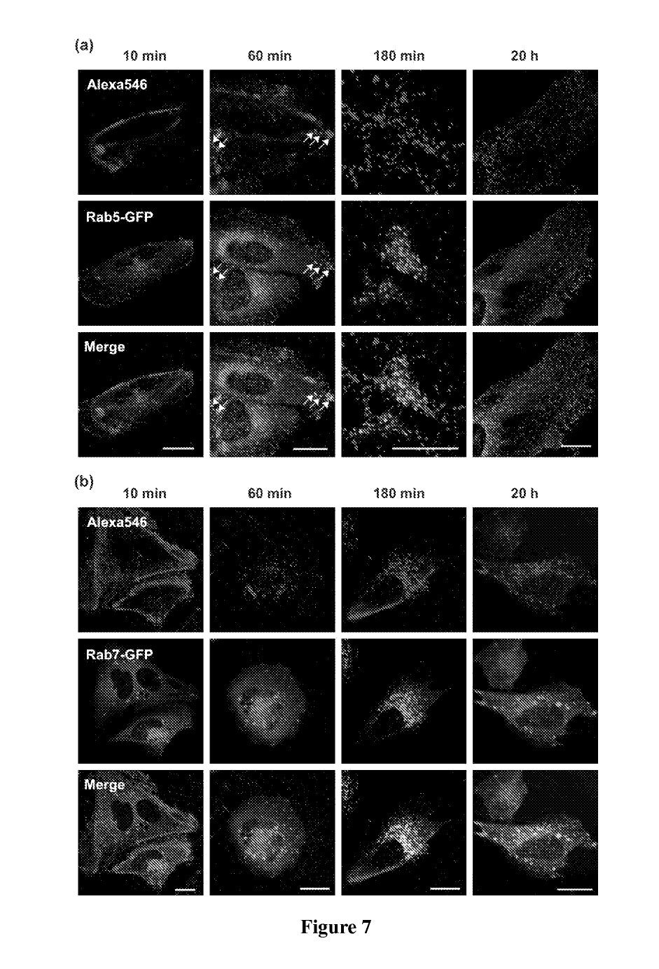

To test the ability of our nanoparticles to deliver cargo to the cytosol, we used either surface biotinylation of live LN-229 cells (human glioma, ATCC) in combination with streptavidin-functionalized NPs, or PEI-PEG-coated NPs to mediate cell surface attachment. Both types of particles are efficiently internalized by cells, and localize to the endo-lysosomal compartment after 3 hours of incubation at 37° C. (FIG. 6). To characterize the internalization pathway, LN-229 cells were transiently transfected with a GFP fusion protein of the lysosomal marker LAMP1, incubated at room temperature with PEI-PEG coated NPs (30 min at 20 ug/mL), followed by removal of unbound particles, and incubation at 37° C. Cells were then imaged at various time points to monitor NP internalization (FIG. 6a). While exclusively present on the cell surface at early time points, the NPs are progressively internalized and fully colocalize with LAMP-1 vesicles for incubations lasting at least 3 hours at 37° C. While all internalized NPs localize to the endo-lysosomal compartment, some patches of surface bound particles can still be observed after extensive incubation periods in some cells (FIG. 6a, bottom row). Similar experiments were also performed using the early endosome marker RabS and late endosome marker Rab7. Exemplary results are summarized in FIG. 7. Streptavidin-functionalized NPs were also found to localize to the endo-lysosomal compartment after overnight incubation with surface-biotinylated LN-229 cells. The internalized NPs were found to colocalize with LysoTracker Blue, a marker of acidic organelles (FIG. 6b).

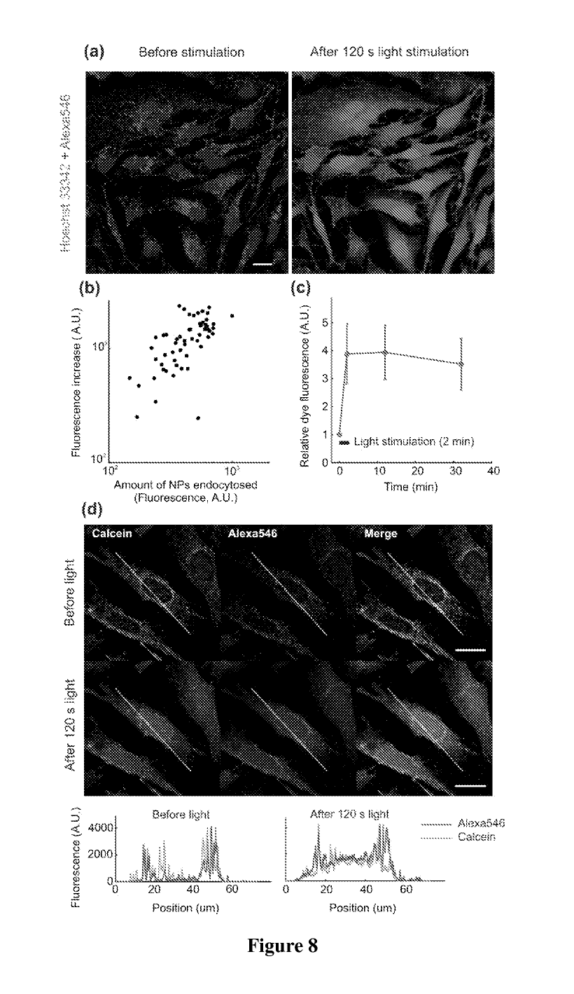

Following overnight NP endocytosis, cells were exposed to green excitation light (520-550 nm, MWIG3/TRITC filter) for durations ranging from 3 s to 120 s. Release into the cytosol was observed immediately after exposure (0.5 mW measured power over the field of view; 500 mW/cm²), for both streptavidin (FIG. 8a) and PEI-PEG coated particles (FIG. 8d), as evidenced by a large increase in fluorescence, especially visible across the nucleus for many cells. Vesicle fluorescence was also increased, which can be explained by a reduction of self-quenching for the unreleased cargo as the highly concentrated fluorophore escapes from the lysosomes. Without being bound to any particular theory, it is contemplated that this light-induced cytosolic release is due to endosomal membrane damage mediated by ROS (e.g., singlet oxygen) produced by a photosensitizer (here Alexa 546) during illumination. The amount of dye released in the cytosol following light exposure was found to be proportional to the number of NPs internalized by cells (FIG. 8b). Dye transfer from endosomes to cytosol is rapid, which is compatible with diffusion kinetics of a small molecule like Alexa 546 (FIG. 8c), and suggests that the NPs themselves are not required to move into the cytosolic compartment for the effect to occur. When PEI-PEG coated NPs were incubated overnight with LN-229 cells in the presence of an excess of calcein, exposure of the cells to green light caused cytosolic release of both the NP dye cargo and co-endocytosed calcein, as evidenced by strong nuclear accumulation of the compounds (see fluorescence profile plots, FIG. 8d).

Confocal micrographs were analyzed using the software package Image-J. Average cell fluorescence was determined by manual contouring of cell borders. Data were then imported into Matlab (The Mathworks) for analysis and plotting purposes. The amount of dye released in the cell cytosol following light stimulation was taken as the difference in average cell fluorescence before and after light exposure.

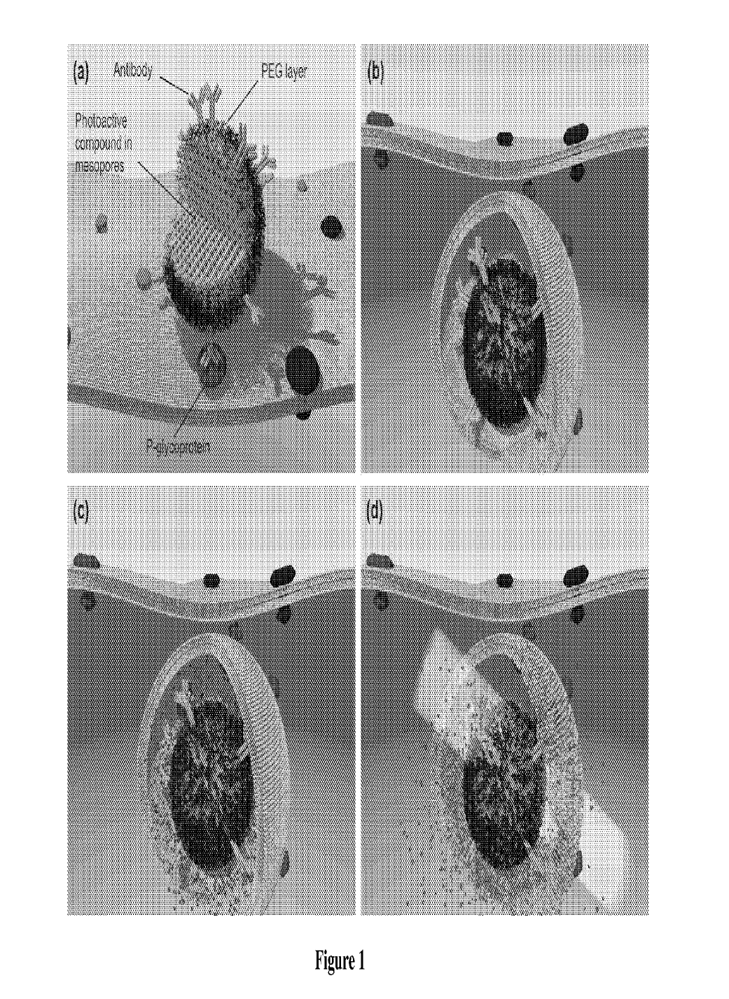

By exploiting the photoactive properties of the model drug in the example, Alexa 546, the timing of cargo release could be precisely controlled by light-stimulated, reactive oxygen species (ROS)-mediated endosomal disruption (as illustrated in FIG. 1).

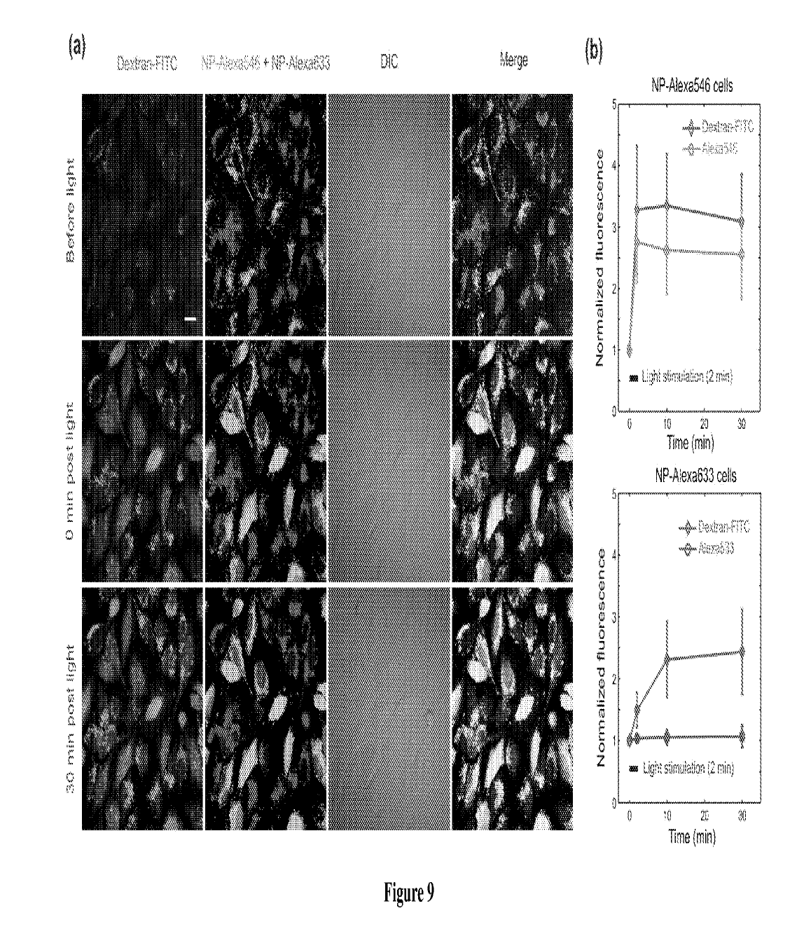

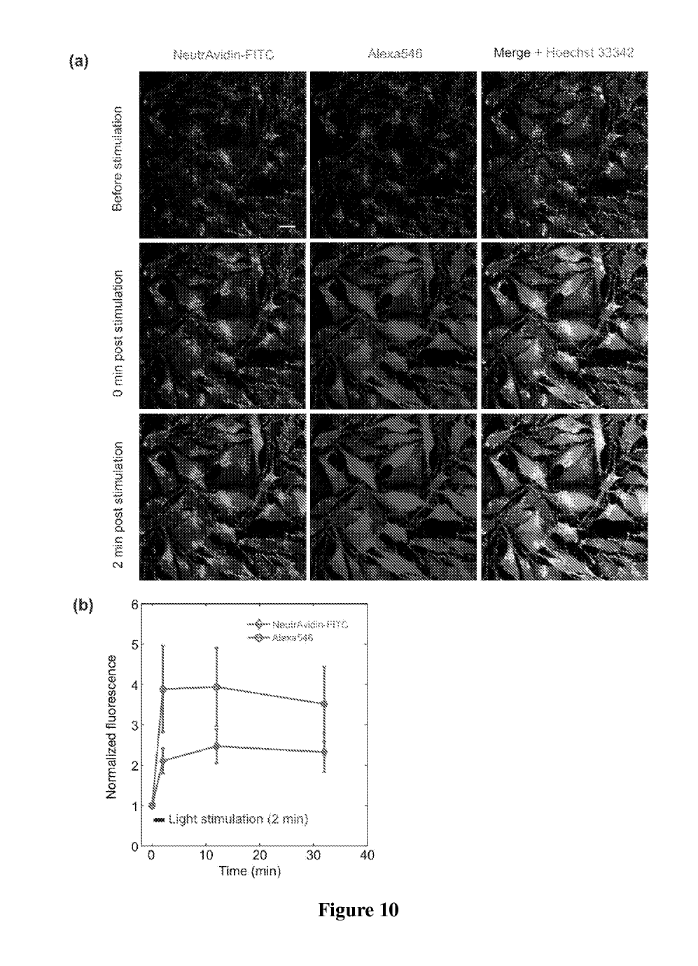

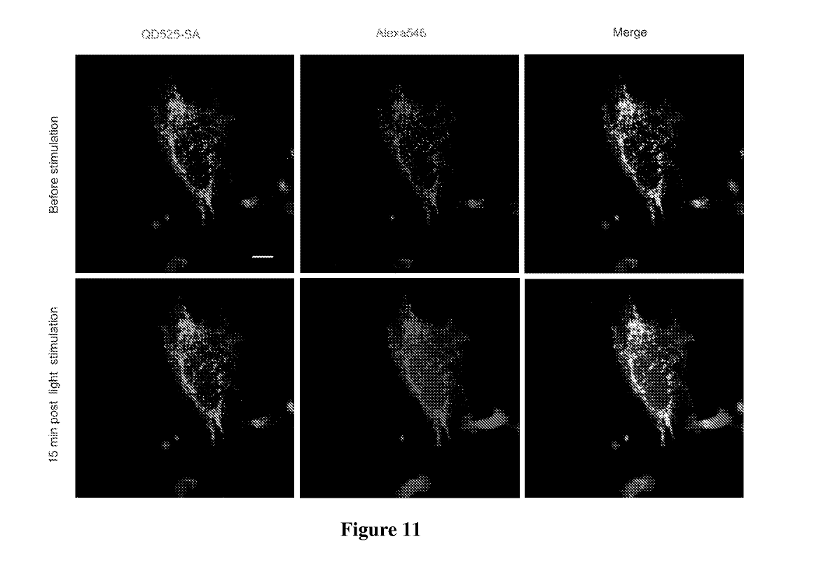

We then determined whether the NPs could liberate co-endocytosed macromolecules and nanoparticles. The NPs successfully mediated cytosolic delivery of a 3 kDa dextran-FITC conjugate (FIG. 9). In this experiment, one set of LN-229 cells was incubated with NPs loaded with a releasable cargo dye (Alexa 546), while a second set was incubated with NPs carrying a dye covalently bound to their matrix during synthesis (Alexa633; so it could not be released). The two cells populations were then plated together, and incubated in the presence of dextran-FITC. Illumination of this mixed population gave the results summarized in FIG. 9. Both types of NPs successfully mediated cytosolic delivery of the FITC-dextran macromolecules, albeit with different kinetics. The lower release kinetics of dextran in the case of the covalently labeled Alexa633-NPs may be explained by a lower excitation efficiency of this fluorophore through the TRITC filter set used for exposure. Covalently labeled Alexa 633-NPs allowed successful release of dextran upon illumination; yet, no Alexa 633 signal was observed in the cytosol after >30 min following exposure to light. These results indicate that the NPs themselves remained confined inside endosomes even after their rupture. Proteins such as NeutrAvidin could also be delivered to the cell cytosol, with slower diffusion kinetics than a dye molecule (FIG. 10). However, co-endocytosed quantum-dot-streptavidin conjugates remained clustered in the endo-lysosomal compartment following light activation (FIG. 11).

For experiments involving the Rab-5, Rab-7 and Lamp-1 markers, LN-229 cells (ATCC) were transfected with the corresponding GFP fusion construct using Lipofectamine 2000 (Invitrogen) according to the manufacturer's instructions and propagated in DMEM supplemented with penicillin/streptomycin, 10% fetal bovine serum. One day post-transfection, cells were trypsinized and re-plated on glass coverslips. After 24 hours, the coverslips were washed ×3 in D-PBS, and incubated with 20 ug/mL PEI-PEG NPs in D-PBS for 30 min at room temperature. The coverslips were then washed ×3 in D-PBS to remove unbound excess NPs, and incubated at 37° C. with 5% CO₂ in DMEM supplemented with penicillin/streptomycin. The cells were taken out of the incubator at various time points, and quickly washed ×3 in D-PBS before confocal imaging at room temperature (FluoView-1000, Olympus).

For LysoTracker staining, following NP uptake, the cells were incubated with 50 nM LysoTracker Blue DND-22 (Invitrogen) at 37° C. with 5% CO₂ in DMEM supplemented with penicillin/streptomycin. After 30 min of incubation, the coverslips were washed ×3 in D-PBS before confocal imaging at room temperature.

### Example 9

### Dextran, NeutrAvidin, and QD Release

This example further illustrates cytosolic delivery of macromolecules.

LN-229 cells were biotinylated at room temperature with NHS-PEO₄-biotin in D-PBS (500 μM, Pierce) supplemented with 10 mM glucose for 30 min, washed ×5 in D-PBS, and trypsinized in 0.01% trypsin/EDTA. Cells were collected with DMEM-10% FBS, centrifuged for 5 min at 1,000 rpm, and resuspended in a 50 μg/mL solution of dye-loaded NP-streptavidin in D-PBS-glucose. After 30 min, the cells were washed twice in D-PBS, resuspended in DMEM-10% FBS, and plated on 12 mm glass coverslips at a density of ˜10⁵ cells/cm². Dextran-FITC (3 kDa, Sigma), NeutrAvidin-FITC (Pierce), or QD525-streptavidin conjugates (Invitrogen) were added to the cells at respective concentrations of 1 mg/mL, 5 μg/mL, and 2 nM. Cells were placed in a 37° C. incubator overnight with 5% CO₂ to allow for coverslip attachment and simultaneous endocytosis of the surface-bound NP-streptavidin conjugates, as well as dextran, NeutrAvidin, or QDs present in the culture medium. After overnight endocytosis, cells were washed twice in D-PBS, and transferred to a live-cell imaging chamber for light stimulation and observation in D-PBS supplemented with 10 mM glucose. Cells were exposed to light from a mercury arc lamp through a TRITC filter as described in Methods, for durations varying from 30 to 120 s, and subsequent changes in the fluorescence distribution within the cells were observed for 30 min.

For experiments involving two types of NPs, the orange pseudo-colored NPs were the post-synthesis Alexa 546 loaded NPs used in all other experiments, while the red pseudo-colored NPs were covalently bound to Alexa 633 incorporated during synthesis of the NPs (1 μL of MPTMS and 20 μL of a 10 mM stock solution of Alexa 633-maleimide were incubated overnight at room temperature, and premixed with the silicate precursors before injection into the reaction mixture. Unreacted dye was removed during the surfactant extraction process). The “orange” nanoparticles can release part of their cargo, while the dye is irreversibly bound to the silica matrix for the “red” NPs. After trypsinization, the biotinylated LN-229 cells were incubated separately with each type of NP, and remixed at 1:1 before plating and overnight incubation with dextran-FITC.

### Example 10

### P-Glycoprotein Targeting and Light-Activated Release

In a typical targeting experiment, cells were trypsinized, plated onto glass coverslips and grown to ˜70% confluence. After removal from the incubator, coverslips were transferred to a Dulbecco-modified phosphate buffered saline (D-PBS, Gibco-Invitrogen) solution supplemented with 10 mM glucose and kept at room temperature for the duration of the experiment. Coverslips were first stained with mouse-anti-human-P-gp primary antibody (BD) at 2.5 μg/mL in the presence of 1% bovine serum albumin (BSA), followed by a biotinylated goat-anti-mouse secondary antibody (Invitrogen) at 5 μg/mL. Streptavidin-functionalized nanoparticles were diluted at 20-100 μg/mL in D-PBS and applied to antibody stained coverslips for 20-30 min, after which they were transferred back to growing medium and incubated at 37° C. to allow endocytosis. After NP uptake, coverslips were transferred to a live imaging chamber in D-PBS and observed by laser scanning fluorescence confocal microscopy (FluoView-1000, Olympus).

To induce cytosolic release of Alexa546, cells were exposed to green excitation light from a mercury arc lamp through a TRITC filter and focused by a 60× water-immersion objective onto the field of view, for durations ranging from 3 s to 120 s. Light fluence over the 200 μm×200 μm field of view was measured through a mask with an equally sized pinhole and found to be 500 mW/cm² (ThorLabs PM100D optical power meter, S130VC probe).

### Example 11

### Single Vesicle Disruption

The experiment described in this example demonstrates using laser scanning confocal microscopy to observe single vesicle disruption events. In some embodiments, this technique used in accordance with the present invention offers a combination of features allowing unprecedented control over cytosolic access of a drug in irradiated cells, while preserving unexposed cells.

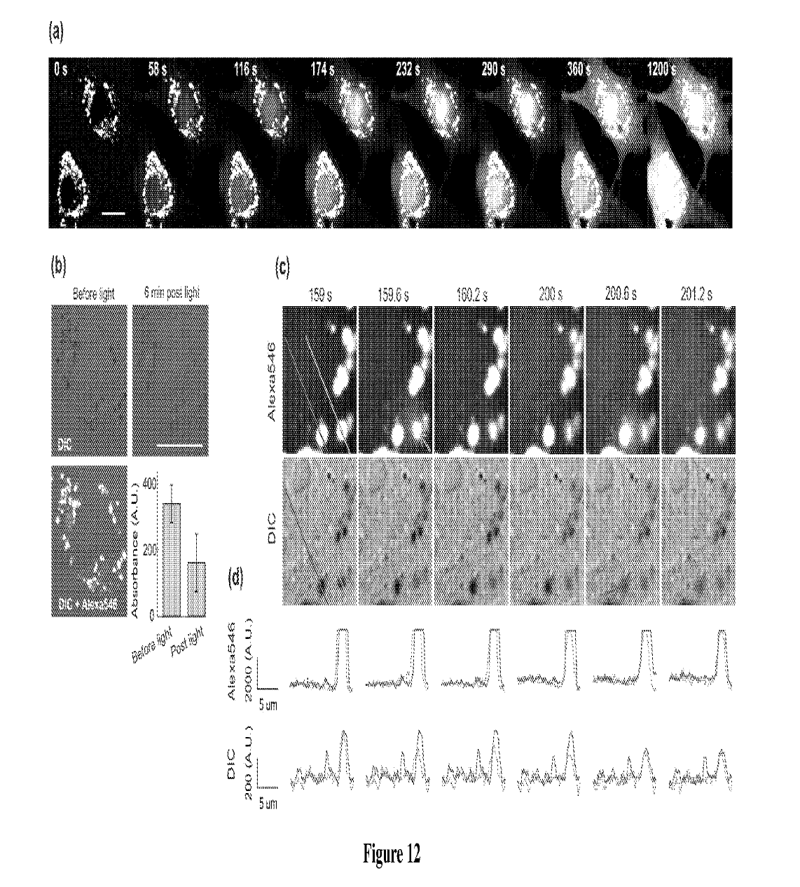

Specifically, incubation of the PEI-PEG coated NPs with bovine aortic endothelial cells (BAEC) resulted in accumulation of the NPs in large lysosomal vesicles, enabling monitoring of single vesicle disruption events following light exposure (FIG. 12). Bovine aorta endothelial cells (BAEC) cells were plated on glass coverslips in DMEM supplemented with 10% FCS. After cell attachment, they were incubated overnight with 20 ug/mL Alexa546-loaded PEI-PEG MSN. Following NP uptake, cells were washed ×3 in D-PBS supplemented with 10% glucose and imaged. Cells were exposed to TRITC-filtered light for 3 s, and monitored under confocal microscopy at fast scanning rates (580 ms/frame) with minimal imaging laser power. Multiple vesicle disruption events could be observed over a period of minutes following light activation. FIG. 12a shows dye fluorescence at various time points up to 20 min, demonstrating progressive cytosolic accumulation of the NP cargo with strong nuclear translocation.

Strikingly, observation of the cells in transmitted light showed a purple color in the intracellular vesicles characteristic of the NP-loaded dye, which was strongly attenuated following cargo release. This can be monitored via DIC microscopy (FIG. 12b), as light absorption by the vesicles (quantified as the difference between average transmittance for the entire image and average transmittance of a given vesicle) significantly decreases following exposure to light. FIG. 12c shows details of NP-loaded lysosomal vesicles following a 3 s exposure to green light (at time 0 s), with successive disruption of two vesicles. Line profile plots of dye fluorescence and absorption (computed from DIC intensity) across these two vesicles are displayed in FIG. 12d. The disruption events are characterized by the appearance of a short-lived fluorescence halo surrounding them as dye rapidly diffuses into the cell cytosol, accompanied by a concomitant drop in absorption within the vesicles (which is directly proportional to dye concentration). Multiple endosome disruptions were observed. When a vesicle breaks in the vicinity of a dendritic process, the diffusing dye is especially noticeable as a fluorescence wave fills up the membrane protrusion, followed by a rapid decrease in signal as the dye diffuses in the entire cell.

### Example 12

### Targeting and Light-Activated Release into MDR Cells

This example demonstrates that nanoparticle-mediated light-triggered cytosolic delivery may be used to specifically target multidrug resistant (MDR) cells.

Almost half of human tumors develop MDR, whereby exposure to a chemotherapeutic agent triggers simultaneous resistance to a wide spectrum of different compounds, even to those to which the cell had never been exposed. In most cases, MDR results from the expression of membrane transporters that actively extrude cell-permeable cytotoxic compounds. The most well characterized MDR transporter is P-glycoprotein (P-gp), a 170 kDa member of the adenosine triphosphate (ATP)-binding cassette (ABC) superfamily that can extrude a wide spectrum of compounds. Such lack of substrate specificity makes this clearance pathway difficult to circumvent. As our method allows cytosolic delivery of cell-impermeable compounds, which are generally not P-gp substrates, we believe our technique is a novel approach to bypass MDR.

To study the targetability of the functionalized NPs to P-gp-expressing cells, we induced stable expression of a P-gp-GFP C-terminal fusion protein in wild type LN-229 cells. Specifically, the hMDR1 (ABCB1) coding sequence was amplified by PCR from the pHaMDRwt plasmid (mammalian retroviral expression vector deposited by M. Gottesman in the Addgene database; Addgene plasmid N. 10957) using the forward primer: 5′-TAGCCACCATGGATCTTGAAGGGGAC-3′ (SEQ ID NO: 1) and the reverse primer: 5′-CCTTACCGGTTCCACTTCCCTGGCGCTTTGTTCCAG-3′ (SEQ ID NO: 2). The PCR product was digested with NheI and AgeI, and sub-cloned into the NheI and XmaI restriction sites of the pEGFP-N2 mammalian expression vector (Clontech).

LN-229 cells (ATCC) were transfected (Lipofectamine 2000, Invitrogen) with the P-Glycoprotein-GFP construct and propagated in DMEM supplemented with penicillin/streptomycin, 10% fetal bovine serum, and 1 mg/mL neomycin to select for stably expressing cells.

Selection of the MDR phenotype was performed by incubation with the mitochondrial marker tetramethylrhodamine esther (TMRE, Sigma), a known substrate of P-glycoprotein, for 20 min (50 nM in complete medium), followed by FACS sorting (FACSCalibur, BD) to select GFP-positive and TMRE-negative populations. After this round of selection (which produced high- and low-P-gp-GFP-expressing cells), the population was propagated in the same medium, supplemented with 0.5 mg/mL neomycin.

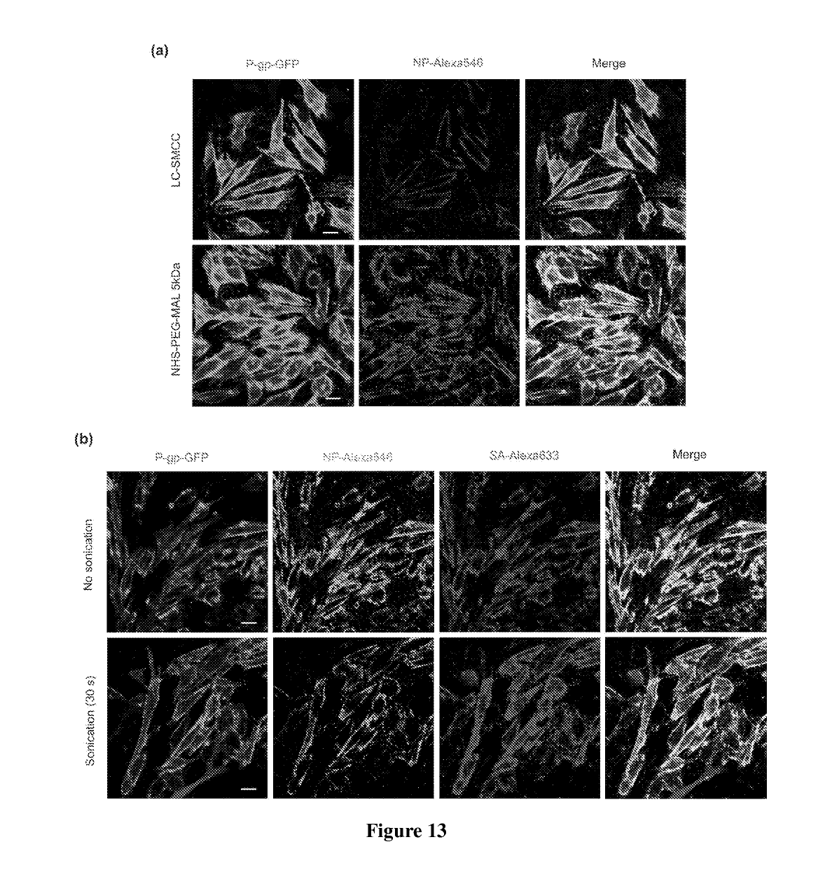

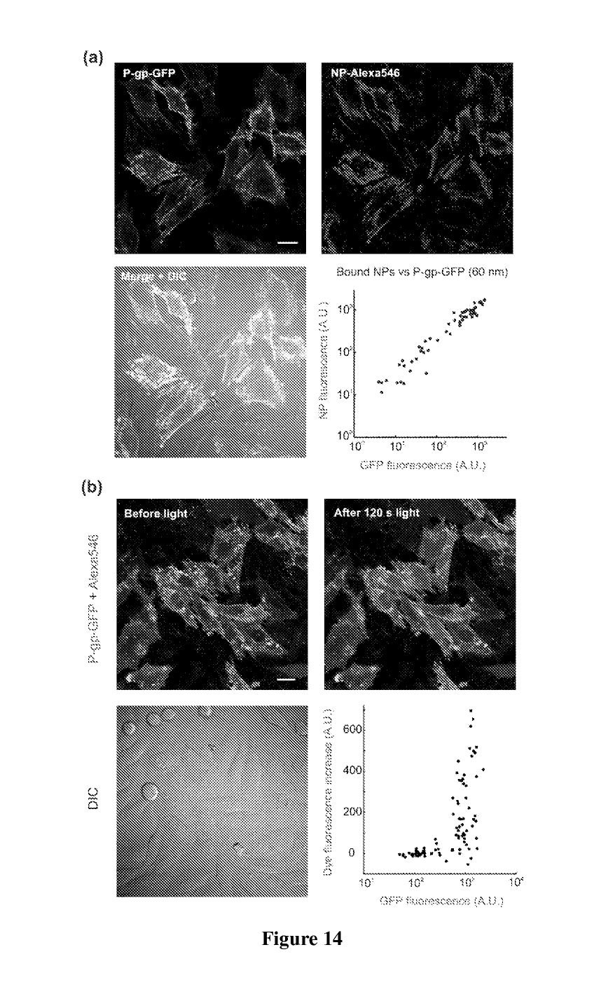

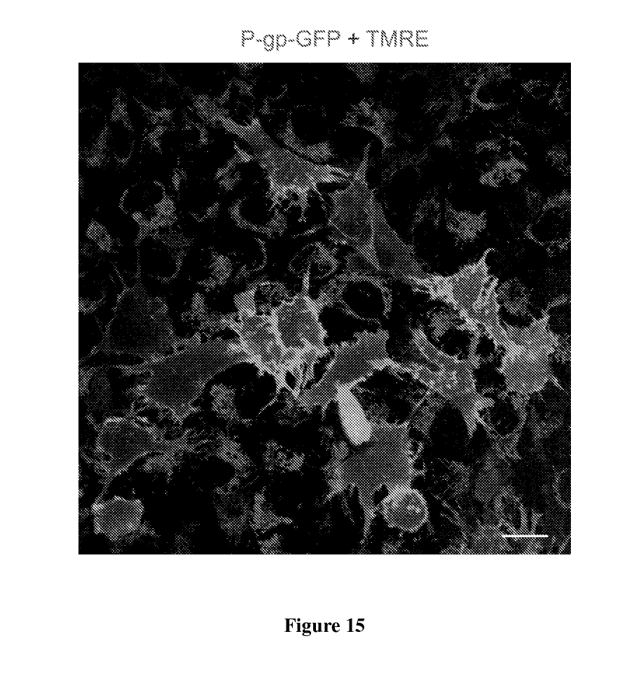

We ascertained P-gp-GFP localization and function by antibody staining and efflux of known substrates, such as rhodamine 123, tetra-methyl-rhodamine-esther, and JC-1 (FIG. 13). Our streptavidin-NP conjugates were successfully targeted to P-gp expressing cells following antibody staining (FIG. 14a). P-gp positive cells showed high levels of NP binding after 20 min of incubation, with few non-specific interactions (FIG. 14a, bottom right panel). As noted above, bioconjugation using a long arm (5 kDa) PEG crosslinker was essential to maximize targetability of the NPs. Shorter arm crosslinkers (such as LC-SMCC, Pierce Biotechnology) also allowed covalent grafting of streptavidin, but resulted in reduced labeling efficiency of cell surface receptors, presumably due to lower mobility of the attached streptavidin as well as lower binding site accessibility (FIG. 15). The use of size exclusion chromatography for conjugate purification also proved important. This method was superior to repeated centrifugation and resuspension steps by sonication, which were found to significantly decrease bioactivity of the immobilized streptavidin (FIG. 15). Moreover, repeated centrifugation steps contributed to the generation of particles aggregates, while column purification did not increase the average hydrodynamic diameter of the NPs.

Following overnight endocytosis of the membrane-bound NPs, regions were chosen that included both wild type cells and cells expressing P-gp. Due to the high specificity of NP uptake, illumination of the entire field released dye exclusively in the P-gp-expressing cells (FIG. 14b). While the amount of cytosol-released dye does not linearly correlate with P-gp expression, there appears to be a threshold beyond which significant release is observed, as evidenced when plotting the increase in dye fluorescence within individual cells following light exposure as a function of their P-gp expression level (FIG. 14b, bottom right panel). The few cells showing a decrease in total cell fluorescence are cells that did not display cargo release, and for which the slight reduction in NP fluorescence can be attributed to either photobleaching or a small focus drift during the course of the experiment.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A composition for targeted drug delivery comprising
nanoparticles;
a targeting moiety specific for a cell type of interest;
a light-activated drug delivery system;
wherein the nanoparticles are associated with the targeting moiety and the light-activated drug delivery system.

2. The composition of claim 1, wherein the nanoparticles are of mesoporous silicate materials.

3. The composition of claim 1, wherein the nanoparticles have size less than 200 nm in diameter.

4-8. (canceled)

9. The composition of claim 1, wherein the nanoparticles are PEGylated.

10. The composition of claim 1, wherein the targeting moiety comprises an antibody or fragment thereof.

11. The composition of claim 10, wherein the antibody or fragment thereof is tumor-specific.

12. (canceled)

13. The composition of claim 10, wherein the antibody or fragment thereof is an antibody specific to a multidrug resistance transporter.

14. The composition of claim 13, wherein the multidrug resistance transporter is MDR1 (also known as P-glycoprotein), or MRP1.

15. The composition of claim 1, wherein the targeting moiety is conjugated to the nanoparticles.

16. The composition of claim 1, wherein the light-activated drug delivery system comprises a photosensitizer and a therapeutic agent.

17. The composition of claim 16, wherein the photosensitizer is capable of causing permeabilisation of endosome membranes upon light activation.

18. The composition of claim 17, wherein the photosensitizer generates reactive oxygen upon light activation.

19. (canceled)

20. The composition of claim 16, wherein the photosensitizer generates reactive oxygen upon X-ray or UV irradiation.

21. (canceled)

22. The composition of claim 16, wherein the therapeutic agent is a protein, a peptide, a nucleic acid, a chemical compound and/or a small molecule.

23. The composition of claim 22, wherein the protein is an antibody or fragment thereof.

24. The composition of claim 22, wherein the nucleic acid is an oligonucleotide.

25. The composition of claim 24, wherein the oligonucleotide is selected from the group consisting of antisense nucleic acids, ribozymes, siRNA, microRNA, aptamer and combination thereof.

26. The composition of claim 16, wherein the therapeutic agent is an anti-cancer agent.

27. The composition of claim 1, wherein the light-activated drug delivery system is covalently or non-covalently associated with the nanoparticles.

28. A method of treating a disease, disorder or condition comprising
administering into a subject in need of treatment the composition of any one of the proceeding claims; and
exposing a tissue of interest to light.

29-39. (canceled)
